(19) United States
(12) Patent Application Publication
(10) Pub. No.: US 2010/0087488 A1
(43) Pub. Date: Apr. 8, 2010

(54) Physiologically Acceptable Salts of 3-[(2--1-methyl-1H-benzimidazol-5-carbonyl)-pyridin-2-yl-amino]-propionic acid ethyl ester

(75) Inventors: Mihaela Pop, Amsterdam (NL); Peter Sieger, Mititelbiberach (DE); Coen Hoogland, Alkmaar (NL); Gerd Kraemer, Eberhardzell (DE)

Correspondence Address: MICHAEL P. MORRIS;BOEHRINGER INGELHEIM USA CORPORATION, 900 RIDGEBURY ROAD, P. O. BOX 368, RIDGEFIELD, CT, 06877-0368, US

(73) Assignee: BOEHRINGER INGELHEIM INTERNATIONAL GMGH, Ingelheim (DE)

(21) Appl. No.: 12/444,762

(22) PCT Filed: Oct. 9, 2007
(86) PCT No.: PCT/EP2007/060711

§ 371 Date: Dec. 15, 2009

(30) Foreign Application Priority Data
Oct. 10, 2006 (EP) 06122047.1

(51) Int. Cl. A61K 31/4439; C07D 401/10; A61P 9/10; A61P 7/02

(52) U.S. Cl. 514/338; 546/273.4

(57) ABSTRACT

The invention relates to new salt forms of the active substance ethyl 3-[(2-{[4-(hexyloxycarbonylamino-imino-methyl)-phenylamino]-methyl}-methyl-1H-benzimidazole-5-carbonyl)-pyridin-2-yl-amino]-propionate.

The invention relates to new salt forms of the active substance ethyl 3-[(2-{[4-(hexyloxycarbonylamino-imino-methyl)-phenylamino]-methyl}-1-methyl-1H-benzimidazole-5-carbonyl)-pyridin-2-yl-amino]propionate, the polymorphs, the enantiomers, the mixtures and the hydrates thereof. This active substance with the chemical formula

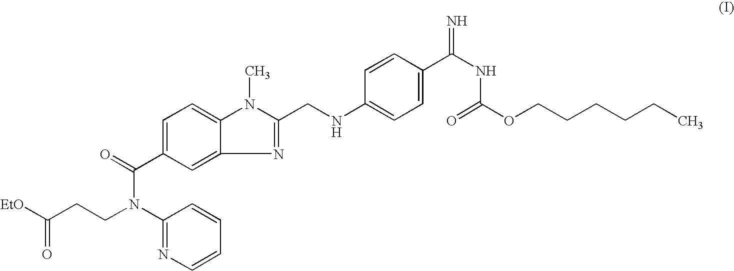

is already known from WO 98/37075, wherein compounds with a thrombin-inhibiting and thrombin time-prolonging activity are disclosed, under the name 1-methyl-2-[N-[4-(N-n-hexyloxycarbonylamidino)phenyl]-amino-methyl]-benzimidazol-5-yl-carboxylic acid-N-(2-pyridyl)-N-(2-ethoxycarbonylethyl)-amide. The compound of formula (I) is also known as BIBR 1048. The compound of formula I is a double prodrug of the compound

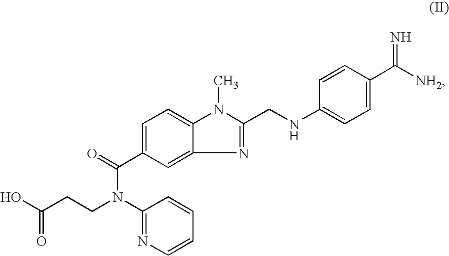

i.e. the compound of formula I is first converted into the actual effective compound, namely the compound of formula II, in the body. The main type of indication for the compound of chemical formula I is the post-operative prophylaxis of deep vein thrombosis and the prevention of strokes.

The aim of the invention is to prepare new salts of the compound of formula I with advantageous properties for pharmaceutical use.

In addition to being effective for the desired indication, an active substance must also conform to additional requirements in order to be allowed to be used as a pharmaceutical composition. These parameters are to a large extent connected with the physicochemical nature of the active substance.

Without being restrictive, examples of these parameters are the stability of effect of the starting material under various environmental conditions, stability during production of the pharmaceutical formulation and stability in the final medicament compositions. The pharmaceutically active substance used for preparing the pharmaceutical compositions should therefore have a high stability which must be guaranteed even under various environmental conditions. This is absolutely essential to prevent the use of pharmaceutical compositions which contain, in addition to the actual active substance, breakdown products thereof, for example. In such cases the content of active substance in pharmaceutical formulations might be less than that specified.

The absorption of moisture reduces the content of pharmaceutically active substance on account of the weight gain caused by the uptake of water. Pharmaceutical compositions with a tendency to absorb moisture have to be protected from damp during storage, e.g. by the addition of suitable drying agents or by storing the medicament in a damp-proof environment. In addition, the uptake of moisture can reduce the content of pharmaceutically active substance during manufacture if the medicament is exposed to the environment without being protected from damp in any way. Preferably a pharmaceutically active substance should therefore have only limited hygroscopicity.

As the crystal modification of an active substance is important to the reproducible active substance content of a preparation, there is a need to clarify as far as possible any existing polymorphism of an active substance present in crystalline form. If there are different polymorphic modifications of an active substance care must be taken to ensure that the crystalline modification of the substance does not change in the pharmaceutical preparation later produced from it. Otherwise, this could have a harmful effect on the reproducible potency of the drug. Against this background, active substances characterised by only slight polymorphism are preferred.

Another criterion which may be of exceptional importance under certain circumstances depending on the choice of formulation or the choice of manufacturing process is the solubility of the active substance. If for example pharmaceutical solutions are prepared (e.g. for infusions) it is essential that the active substance should be sufficiently soluble in physiologically acceptable solvents. It is also very important for drugs which are to be taken orally that the active substance should be sufficiently soluble.

The problem of the present invention is to provide a pharmaceutically active substance which not only is characterised by high pharmacological potency but also satisfies the above-mentioned physicochemical requirements as far as possible.

Surprisingly it has now been found that the salt forms of the compound of formula I (dabigatran etexilate) according to the invention, the polymorphs, the enantiomers, mixtures and hydrates thereof, meet these requirements and have thus advantageous properties.

The invention therefore relates to the salts of ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate with the inorganic and organinc acids listed in table I as “used acid”, as well as the polymorphs, the enantiomers, mixtures, solvates and hydrates thereof. The invention further relates to pharmaceutical compositions containing at least of one of the above-mentioned salts, their polymorphs, hydrates, solvates or co-crystals, and methods of preparing these pharmaceutical compositions which are suitable for the prevention of venous thromboses and stroke.

The salts according to the invention and also ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate in the form of the free base and as a salt with methanesulphonic acid are also suitable for the treatment and prevention of deep vein thromboses in patients with heparin-induced thrombocytopenia and for the prevention of thrombosis in patients with intraarterial or intravenous lines or catheters as well as AV shunts.

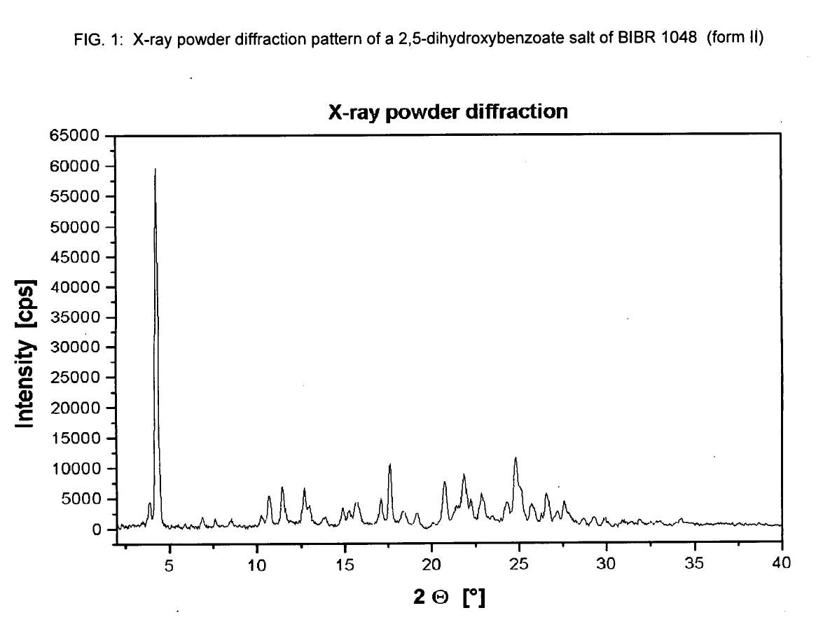

FIGS. 1 to 41 show the X-ray powder diffraction patterns of the salts according to the invention.

The starting compound ethyl 3-[(2-{[4-(amino-hexyloxycarbonylimino-methyl)-phenyl-amino]-methyl}-1-methyl-1H-benzimidazole-5-carbonyl)-pyridin-2-yl-amino]-propionate (BIBR 1048) may for example be prepared as described in International Application WO 98/37075, Example 113.

### General Process for the Preparation of BIBR 1048 Salts

Approx. 750 mg of the free base of BIBR 1048 are dissolved in 10 ml of a mixture of acetone/tetrahydrofuran=80:20. 96 well plates were charged by dosing first this concentrated solution of the free base of BIBR 1048 in acetone/tetrahydrofuran=80:20 and than the respective acids dissolved in water or acetone/tetrahydrofuran=80:20 (for saccharin and salicylic acid). The ratio of BIBR 1048 BS to the respective acid was kept 1:1 for all used acids (see Table I). The plates containing the stock solution were placed in a vacuum chamber (1 kPa) at room temperature for 24 h in order to remove the stock solvent. Afterwards different solvents were added according to table I and the whole 96 well plate is sealed afterwards and heated up with a heating rate of approx. 5° C./min to 50° C. at which the plate stays for an additional 30 minutes. Afterwards the plate is cooled with a cooling rate of 1° C./h, 2° C./h, 3° C./h or 30° C./h to a final temperature of 3, 5, 20 or 25° C. At this temperature the plate remained for a holding time of 1 h, 24 h or 72 h. The plates are opened afterwards and the solids were obtained by filtration.

TABLE I
Conditions for the preparation of the different BIBR 1048 salts

| salt | | | | | cooling | | |
|---|---|---|---|---|---|---|---|
| form | salt form | | ratio | crystallisation | rate | T_{final} | holding |
| abbrev. | full name | used acid | base/acid | solvent | [° C./min] | [° C.] | time [h] |
| Gen2 | 2,5- | 2,5-dihydroxybenzoic | 1:1 | 2-propanol | 2 | 20 | 24 |
| | dihydroxy- | acid | | | | | |
| | benzoate | (gentisic acid) | | | | | |
| | form II | | | | | | |
| Bes1 | besylate, | benzenesulfonic acid | 1:1 | tetrahydrofuran | 3 | 3 | 72 |
| | form I | | | | | | |
| Bes2 | besylate, | benzenesulfonic acid | 1:1 | ethylformate/methanol = | 3 | 3 | 72 |
| | form II | | | 90:10 | | | |
| Bes3 | besylate, | benzenesulfonic acid | 1:1 | nitromethane/acetone = | 30 | 3 | 72 |
| | form III | | | 90:10 | | | |
| HCl2 | chloride, | hydrochloric acid | 1:1 | 1,2- | 1 | 25 | 24 |
| | form II | | | dimethoxy- | | | |
| | | | | ethane | | | |
| HCl5 | chloride, | hydrochloric acid | 1:1 | isopropylacetate | 1 | 5 | 24 |
| | form V | | | | | | |
| HCl6 | chloride, | hydrochloric acid | 1:1 | Tetrahydro- | 1 | 5 | 24 |
| | form VI | | | furan | | | |
| Cyc1 | cyclamate, | Cyclohexanesulfonamide | 1:1 | diethylether | 2 | 20 | 24 |
| | form I | acid | | | | | |
| | | (cyclamic acid) | | | | | |
| Cyc2 | cyclamate, | Cyclohexanesulfonamide | 1:1 | water | 2 | 20 | 24 |
| | form II | acid | | | | | |
| | | (cyclamic acid) | | | | | |
| Eds1 | edisylate, | ethanedisulfonic acid | 1:1 | acetone | 1 | 5 | 24 |
| | form I | | | | | | |
| Eds2 | edisylate, | ethanedisulfonic acid | 1:1 | Tetrahydro- | 1 | 5 | 24 |
| | form II | | | furan | | | |
| Eds3 | edisylate, | ethanedisulfonic acid | 1:1 | nitromethane | no | 20 | 24 |
| | form III | | | | control | | |
| Eds4 | edisylate, | ethanedisulfonic acid | 1:1 | 4-hydroxy-4- | 3 | 3 | 1 |
| | form IV | | | methyl- | | | |
| | | | | 2-pentanone | | | |
| Eds5 | edisylate, | ethanedisulfonic acid | 1:1 | methyl | 30 | 3 | 72 |
| | form V | | | propionate/methanol = | | | |
| | | | | 80:20 | | | |
| Ets1 | esylate, | ethanesulfonic acid | 1:1 | acetone | 2 | 3 | 24 |
| | form I | | | | | | |
| Fum3 | fumarate, | fumaric acid | 1:1 | tert.- | 2 | 3 | 24 |
| | form III | | | butylmethyl | | | |
| | | | | ether | | | |
| Fum4 | fumarate, | fumaric acid | 1:1 | water | 2 | 3 | 24 |
| | form IV | | | | | | |
| D-Glo1 | D- | D-glucuronic acid | 1:1 | 2-butanone | 2 | 20 | 24 |
| | glucuronate, | | | | | | |
| | form I | | | | | | |
| Gly1 | glycolate, | glycolic acid | 1:1 | 1,4-dioxane | no | 20 | 24 |
| | form I | | | | control | | |
| Gly2 | glycolate, | glycolic acid | 1:1 | 2-butanone/water = | 30 | 3 | 1 |
| | form I | | | 90:10 | | | |
| Gly3 | glycolate, | glycolic acid | 1:1 | methanol/acetone = | 30 | 3 | 72 |
| | form I | | | 50:50 | | | |
| Ise3 | isethionate, | 2-hydroxyethane- | 1:1 | 2-propanol | 2 | 3 | 24 |
| | form III | sulfonic acid | | | | | |
| | | (isethionic acid) | | | | | |
| L-Mal1 | L-malate, | L-malic acid | 1:1 | acetone | 1 | 5 | 24 |
| | form I | | | | | | |
| D-Mal1 | D-malate, | D-malic acid | 1:1 | 2-propanol | 2 | 3 | 24 |
| | form I | | | | | | |
| Man1 | mandelate, | S-(+)-mandelic | 1:1 | n-butylacetate | 2 | 20 | 24 |
| | form I | acid | | | | | |
| Nad1 | naphthalene- | naphthalene-1,5- | 1.1 | water | 2 | 20 | 24 |
| | 1,5- | disulfonic acid | | | | | |
| | disulfonate, | | | | | | |
| | form I | | | | | | |
| Nas1 | naphthalene- | naphthalene-2- | 1:1 | acetone | 2 | 20 | 24 |
| | 2-sulfonate, | sulfonic acid | | | | | |
| | form I | | | | | | |
| Oxa1 | oxalate, form I | oxalic acid | 1:1 | acetonitrile | 1 | 5 | 24 |
| Oxa2 | oxalate, form | oxalic acid | 1:1 | isopropylacetate | 1 | 5 | 24 |
| | II | | | | | | |
| Oxa5 | oxalate, form V | oxalic acid | 1:1 | 2-propanol | 1 | 5 | 24 |
| Pho1 | phosphate, | phosphoric acid | 1:1 | isopropylacetate | 1 | 5 | 24 |
| | form I | | | | | | |
| Pho2 | phosphate, | phosphoric acid | 1:1 | 1,4-dioxane | 1 | 5 | 24 |
| | form I | | | | | | |
| Pro1 | propionate, | propionic acid | 1:1 | ethylacetate | 1 | 25 | 24 |
| | form I | | | | | | |
| Pro2 | propionate, | propionic acid | 1:1 | acetonitril | 1 | 25 | 24 |
| | form II | | | | | | |
| Sac1 | saccharinate, | saccharine | 1:1 | ethylacetate | 1 | 25 | 24 |
| | form I | | | | | | |
| Sac2 | saccharinate, | saccharine | 1:1 | 1,2-dimethoxy- | 1 | 5 | 24 |
| | form II | | | ethane | | | |
| Sac3 | saccharinate, | saccharine | 1:1 | perfluorobenzene | no | 20 | 24 |
| | form III | | | | control | | |
| Sac4 | saccharinate, | saccharine | 1:1 | nitromethane | no | 20 | 24 |
| | form IV | | | | control | | |
| Sac5 | saccharinate, | saccharine | 1:1 | 1,4-dioxane | no | 20 | 24 |
| | form V | | | | control | | |
| Sal2 | salicylate, | salicylic acid | 1:1 | water | 1 | 25 | 24 |
| | form II | | | | | | |
| Sal3 | salicylate, | salicylic acid | 1:1 | dichloromethane | 3 | 3 | 72 |
| | form III | | | | | | |
| Suc1 | succinate, | succinic acid | 1:1 | 2-propanol | 1 | 5 | 24 |
| | form I | | | | | | |
| Suc3 | succinate, | succinic acid | 1:1 | dichloromethane | 30 | 3 | 1 |
| | form III | | | | | | |
| D-Tar1 | D-tartrate, | D-tartaric acid | 1:1 | 1,2-dimethoxy- | 1 | 5 | 24 |
| | form I | | | ethane | | | |
| D-Tar2 | D-tartrate, | D-tartaric acid | 1:1 | water | 1 | 25 | 24 |
| | form II | | | | | | |
| Tos1 | tosylate, form I | p-toluenesulfonic | 1:1 | ethylacetate | 2 | 20 | 24 |
| | | acid | | | | | |
| Tos5 | tosylate, form V | p-toluenesulfonic | 1:1 | 2-butanone | 2 | 3 | 24 |
| | | acid | | | | | |
| Tos6 | tosylate, form | p-toluenesulfonic | 1:1 | acetone | 2 | 20 | 24 |
| | VI | acid | | | | | |
| Tos7 | tosylate, form | p-toluenesulfonic | 1:1 | propylacetate | 20 | 3 | 72 |
| | VII | acid | | | | | |

Analytics:

The harvested crystals were analysed by X-ray powder diffraction and thermal analysis (DSC and in some cases also TGA). The following equipment was used:

X-Ray Powder Diffraction (=XRPD):

XRPD patterns were obtained using a high throughput XRPD set-up. The plates were mounted on a Bruker GADDS diffractometer equipped with a Hi-Star area detector. The diffractometer was calibrated using Silver Behenate for the long d-spacings and corundum for the short d-spacings.

The data collection was carried out at room temperature using monochromatic CuK_{α} radiation in the region of 20 between 1.5 and 41.5°. The diffraction pattern of each well was collected with an exposure time of 3-4 minutes.

Thermal Analysis (DSC and TGA):

Melting properties were obtained from differential scanning calorimetry (=DSC) thermograms recorded on a DSC822e (Mettler-Toledo GmbH, Switzerland). The DSC822e was calibrated for temperature and enthalpy with a small piece of indium (T_{fus}=156.6° C., ΔH_{fus}=28.45 J/g). Samples were sealed in standard 40 μl aluminium pans and heated in the DSC from 25 to 300° C. with a heating rate of 20° C./min. Dry nitrogene gas was used to purge the DSC equipment during measurements at a flow rate of 50 ml/min.

The melting temperature used was the onset temperature of the corresponding melting peak in the DSC diagram. The accuracy of the melting points given is about ±3° C.

The mass loss due to solvent or water loss from the crystals was determined by thermo garvimetric analysis (=TGA). During heating of a sample in a TGA/SDTA851e (Mettler-Toledo GmbH, Switzerland) the weight of the sample was monitored resulting in a weight vs. temperature curve. The TGA/SDTA851e was calibrated for temperature with indium and aluminium. Samples were weighed in 100 μl corundum crucibles and heated in the TGA from 25 to 300° C. with a heating rate of 20° C./min. Dry nitrogene gas was used for purging.

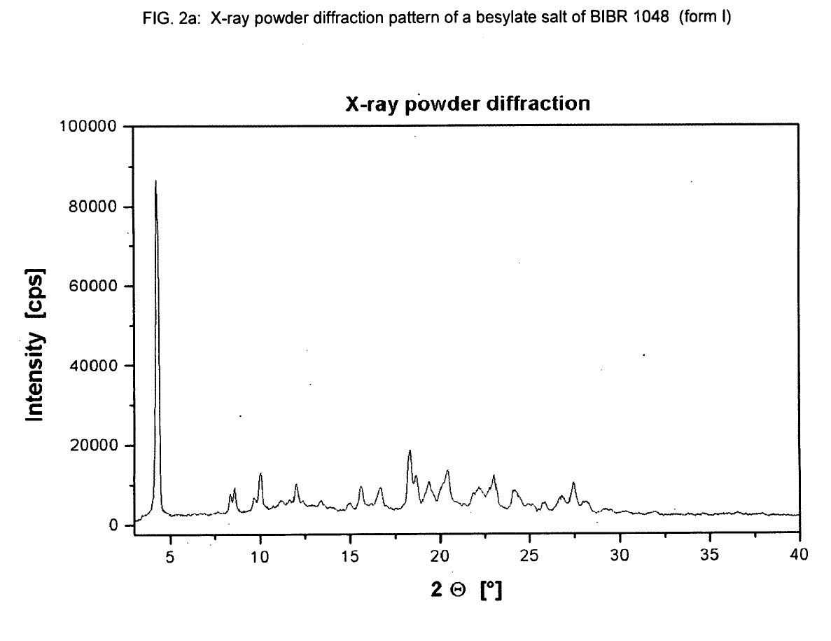

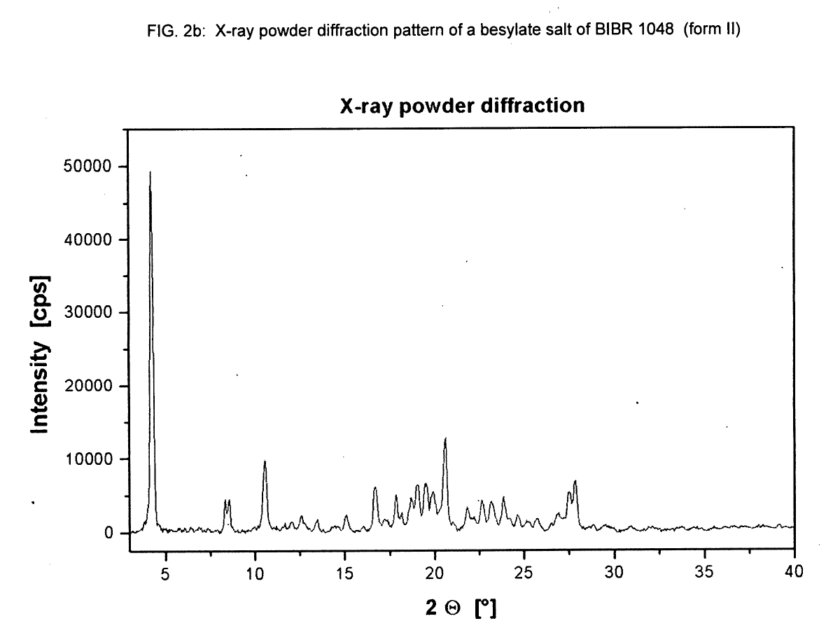

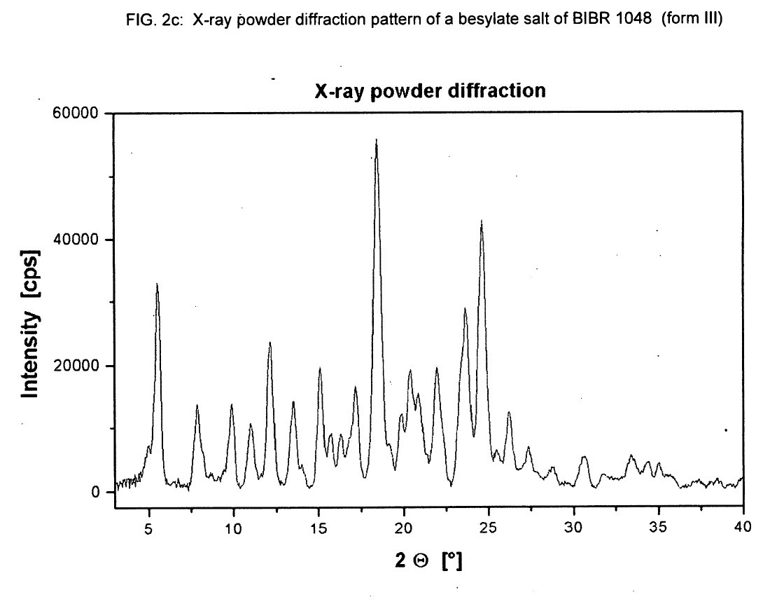

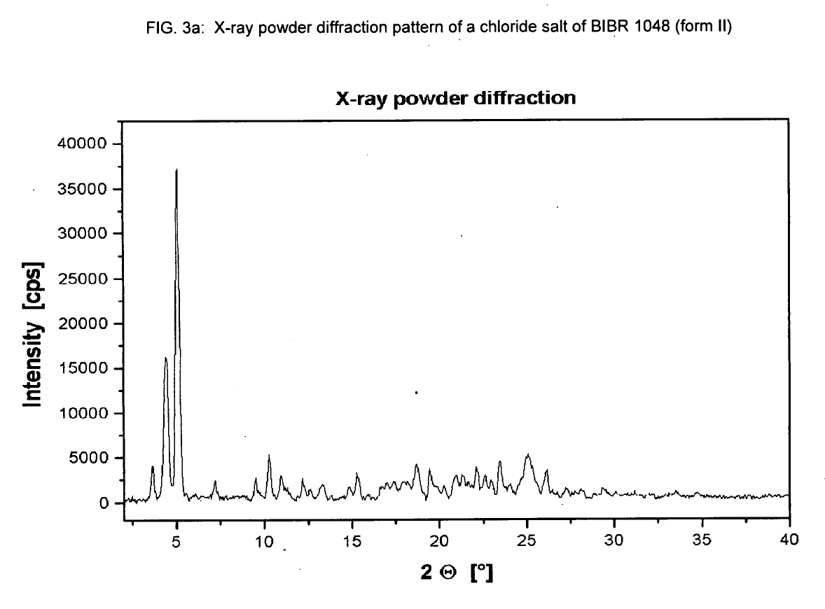

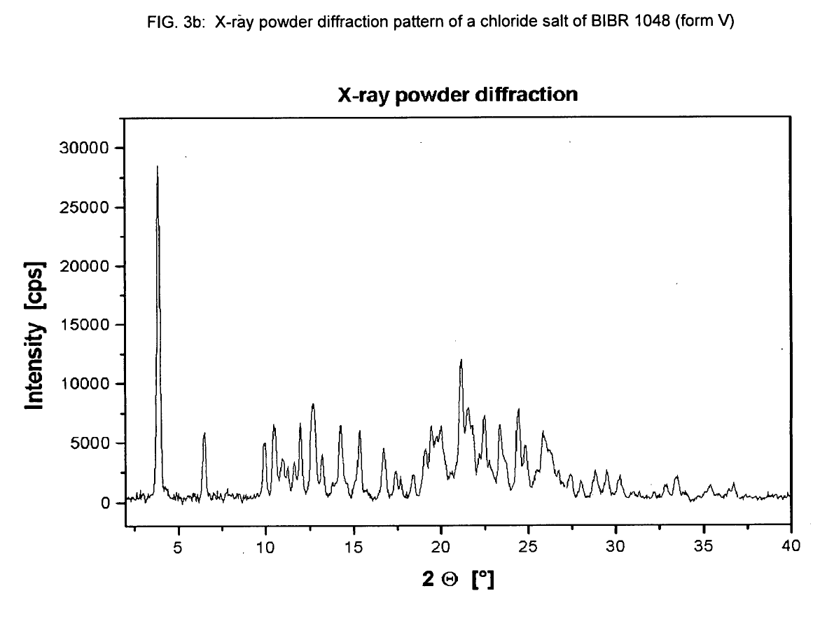

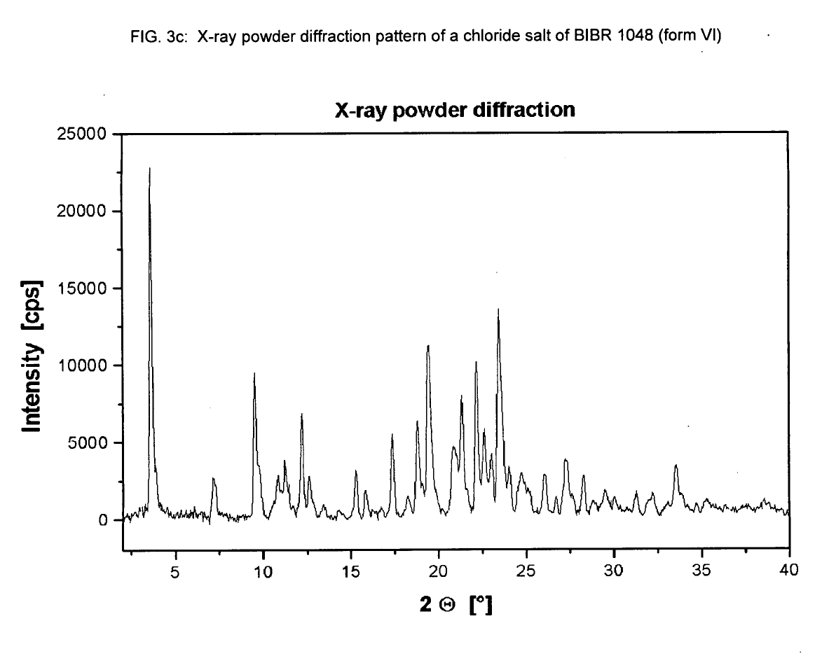

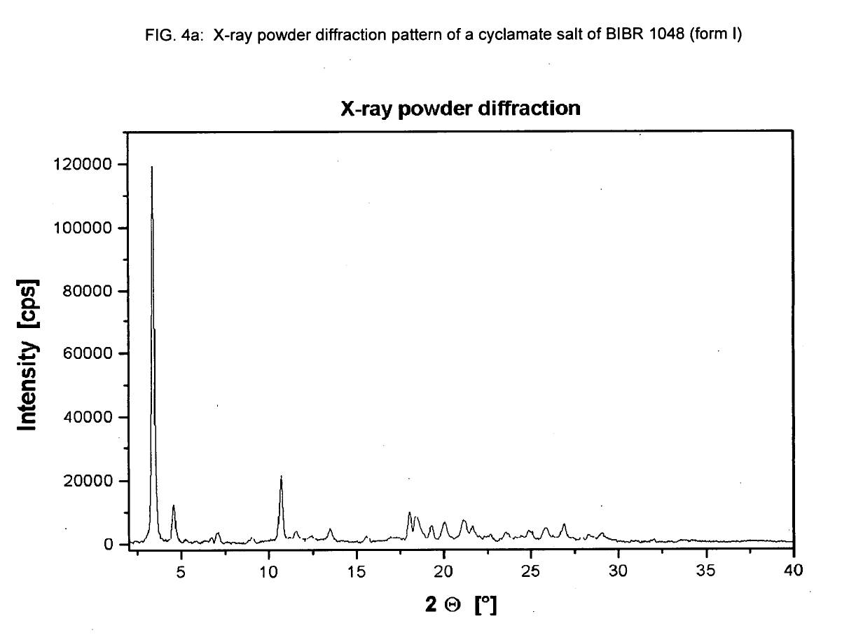

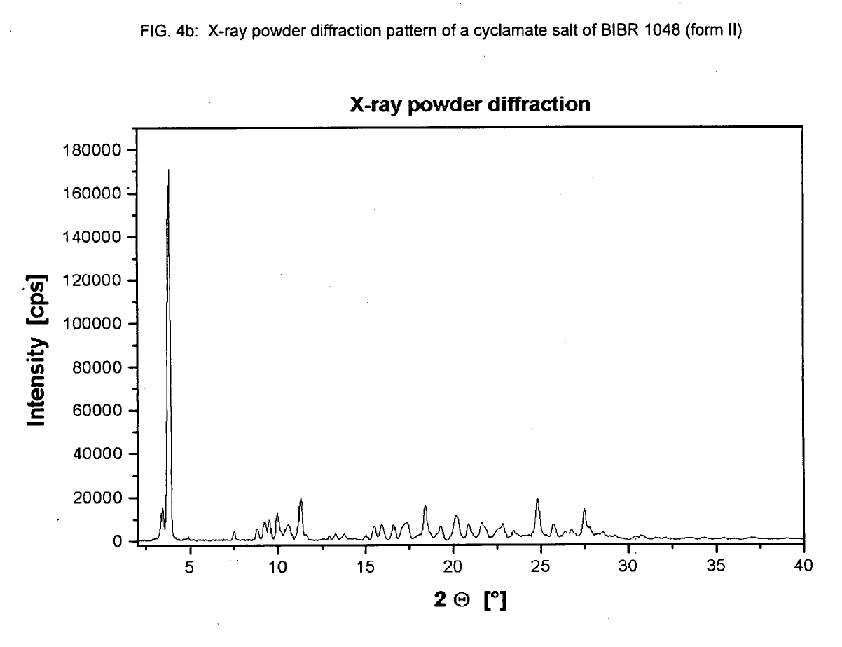

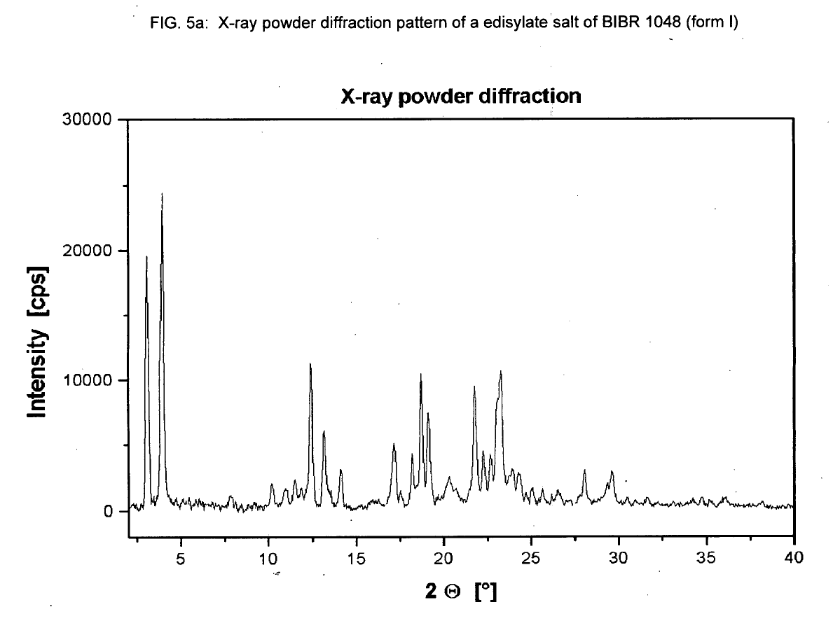

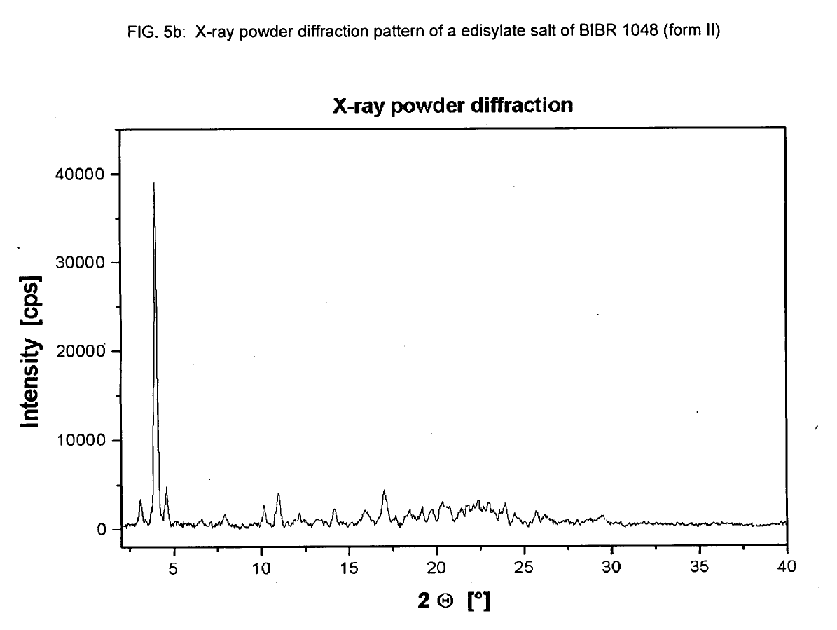

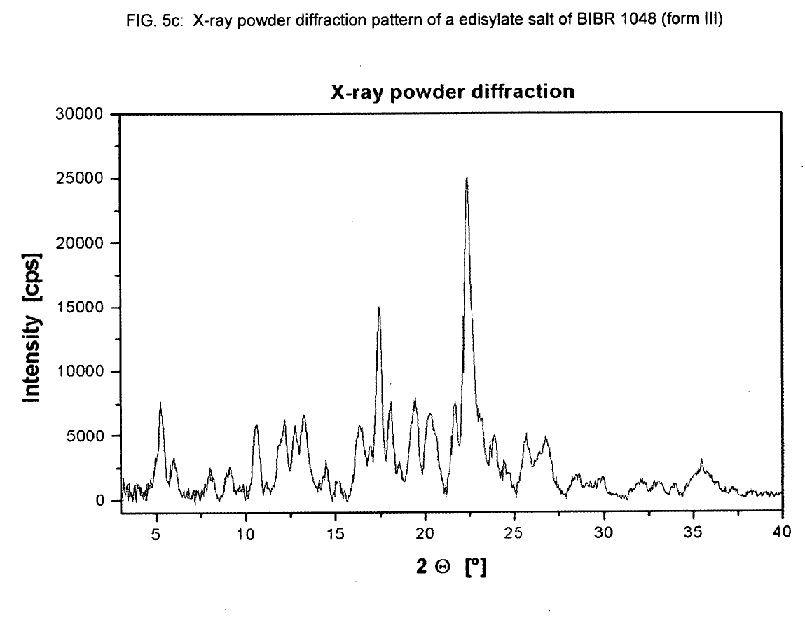

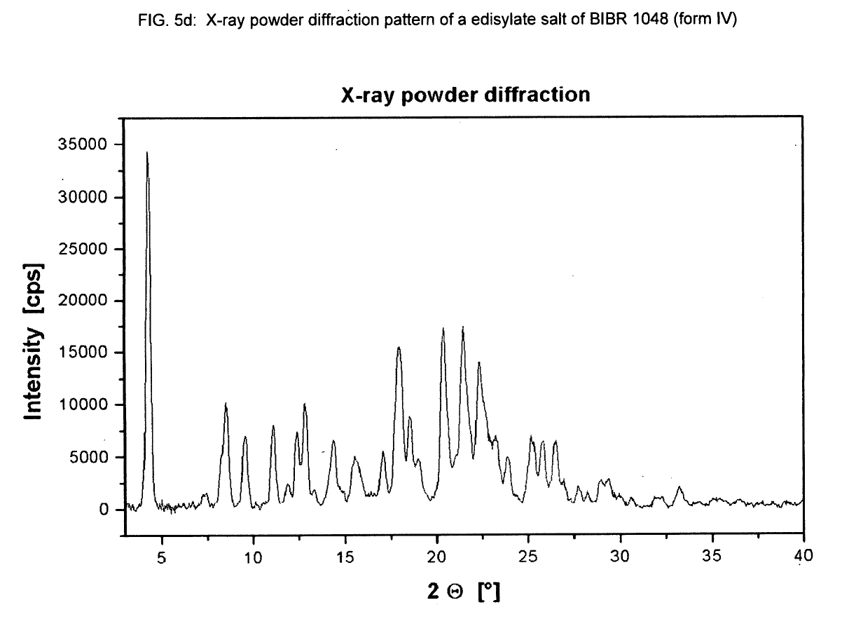

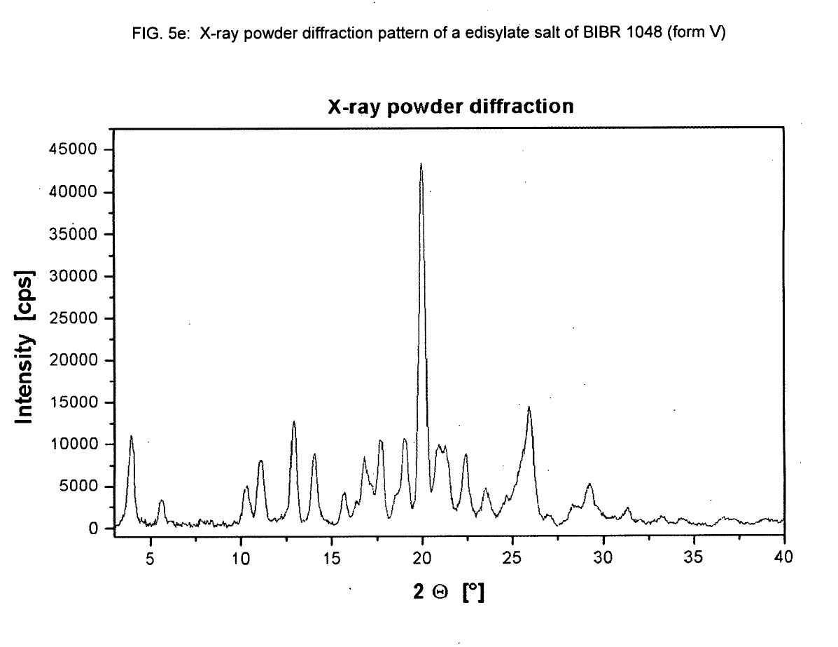

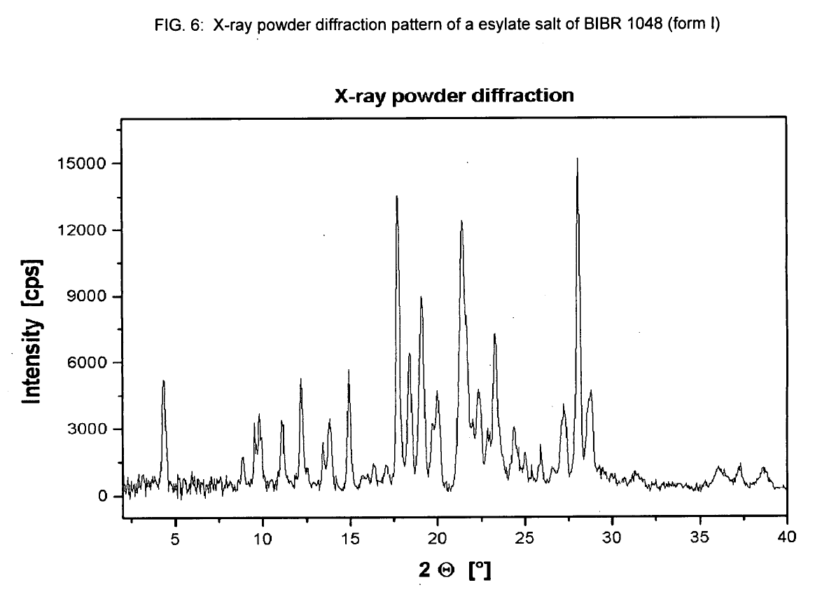

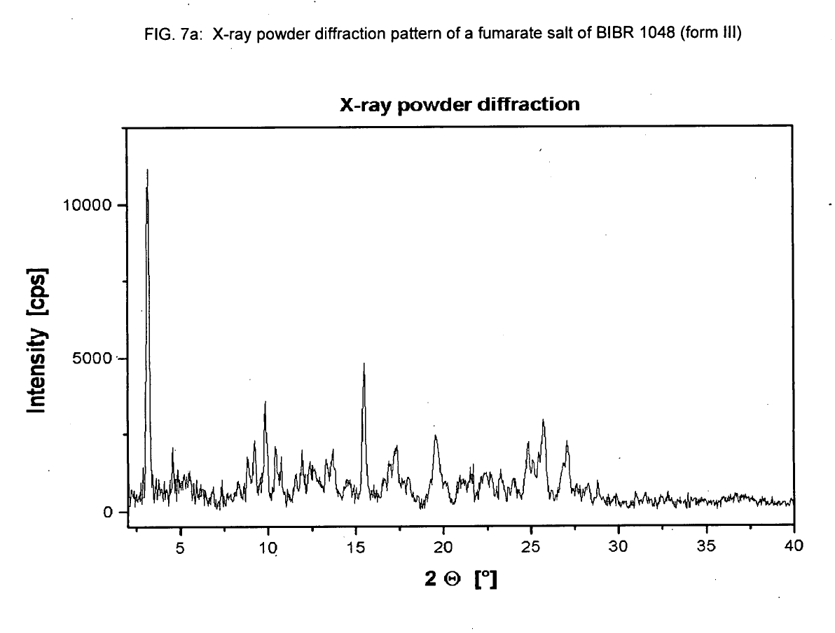

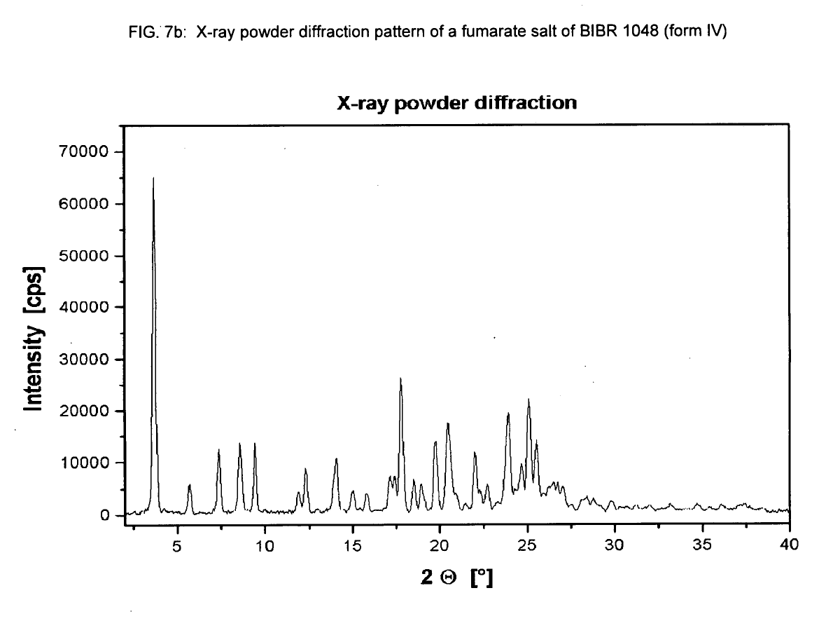

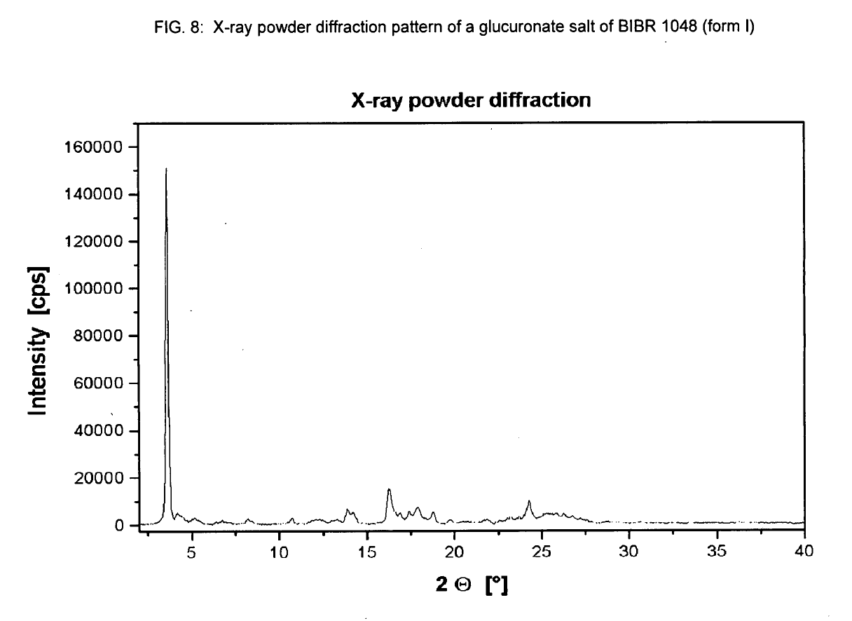

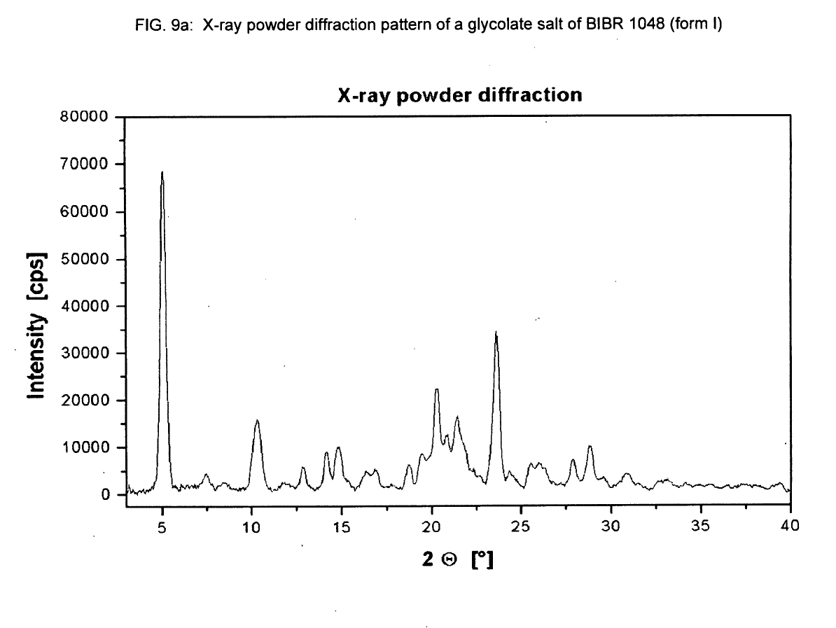

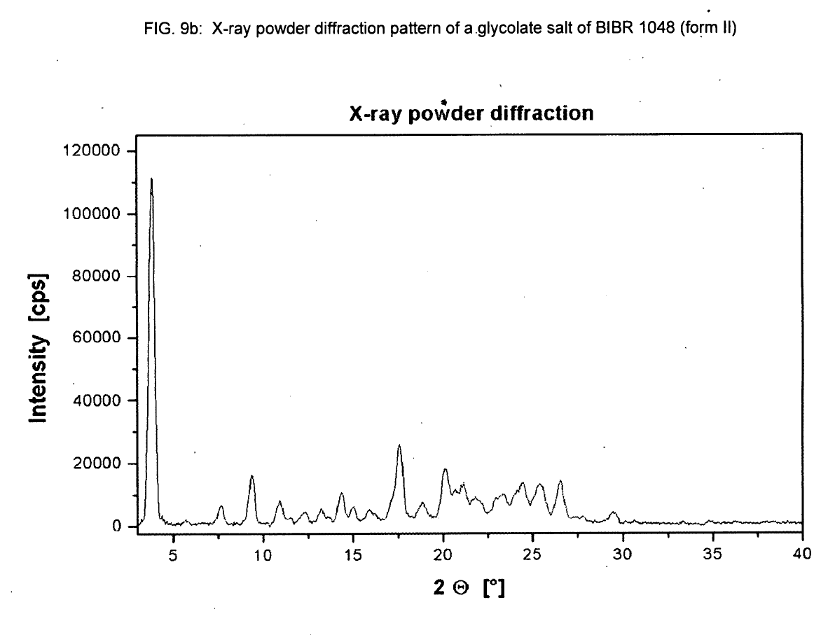

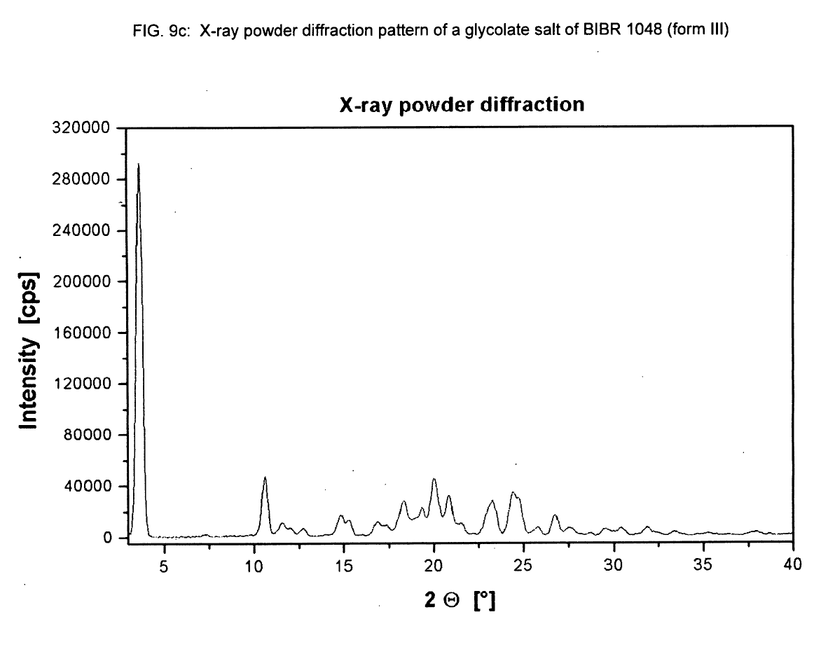

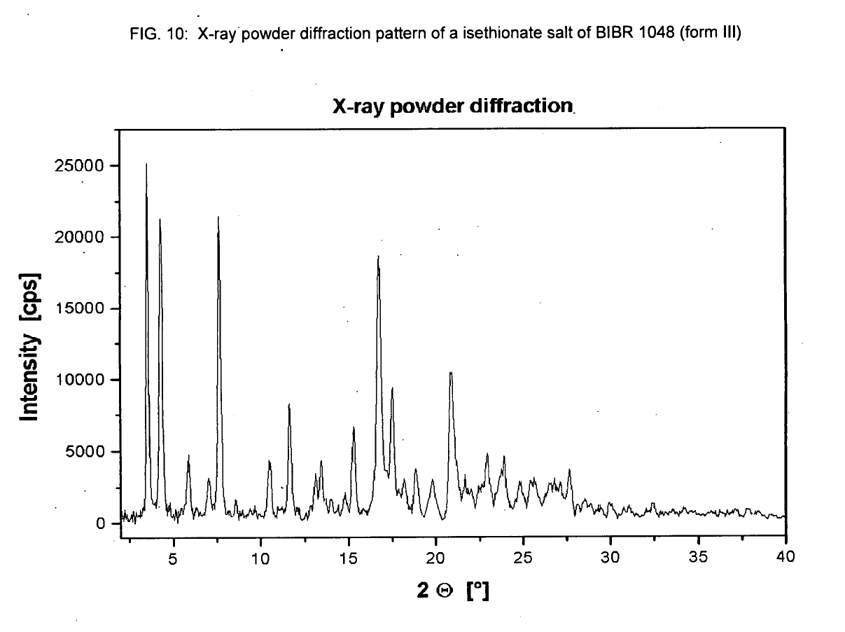

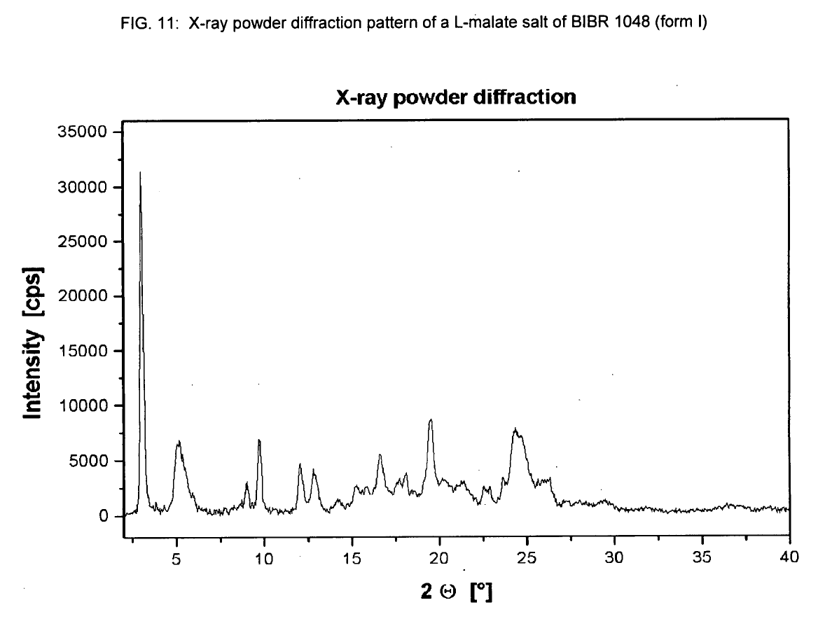

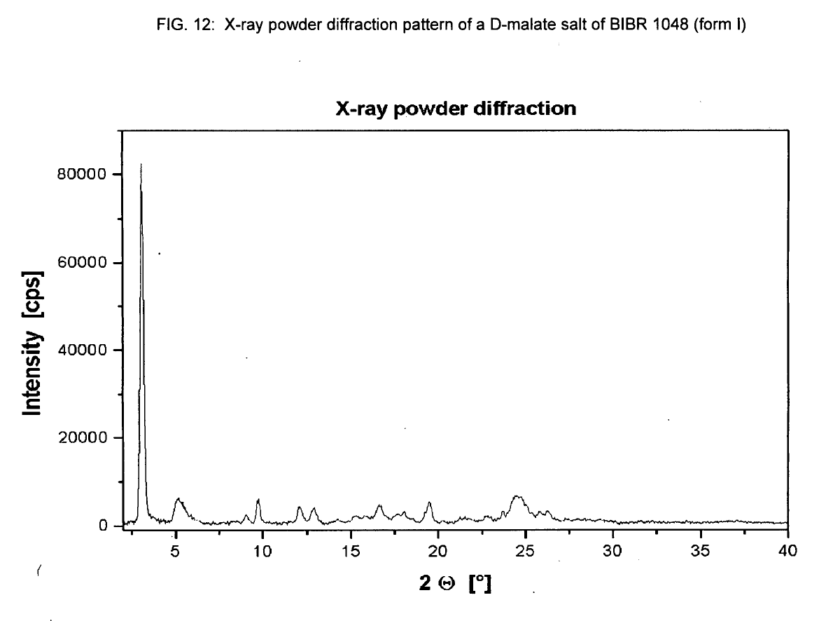

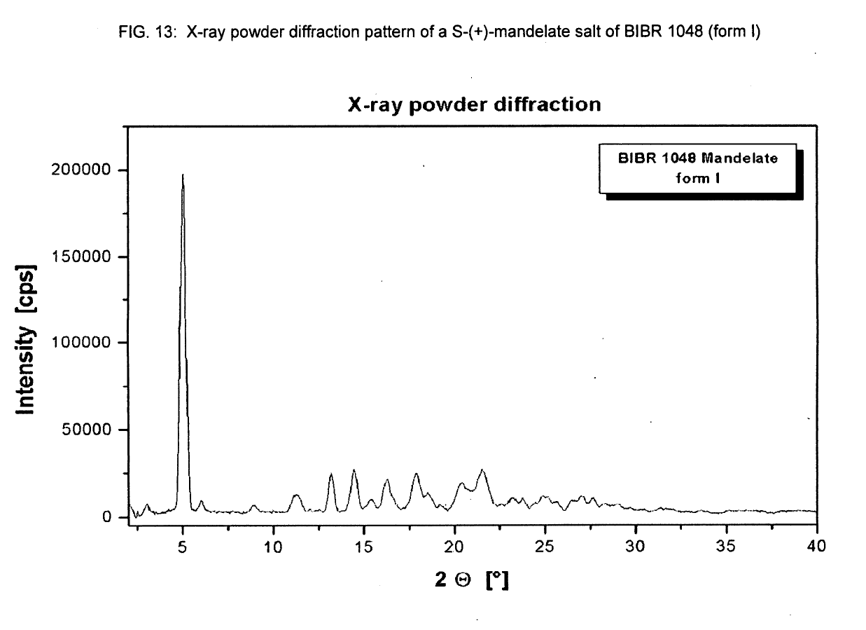

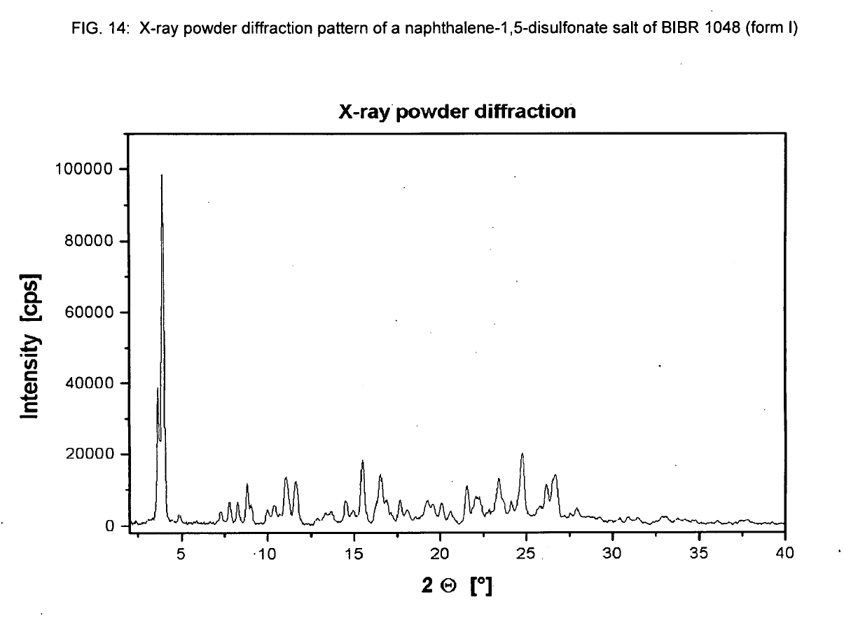

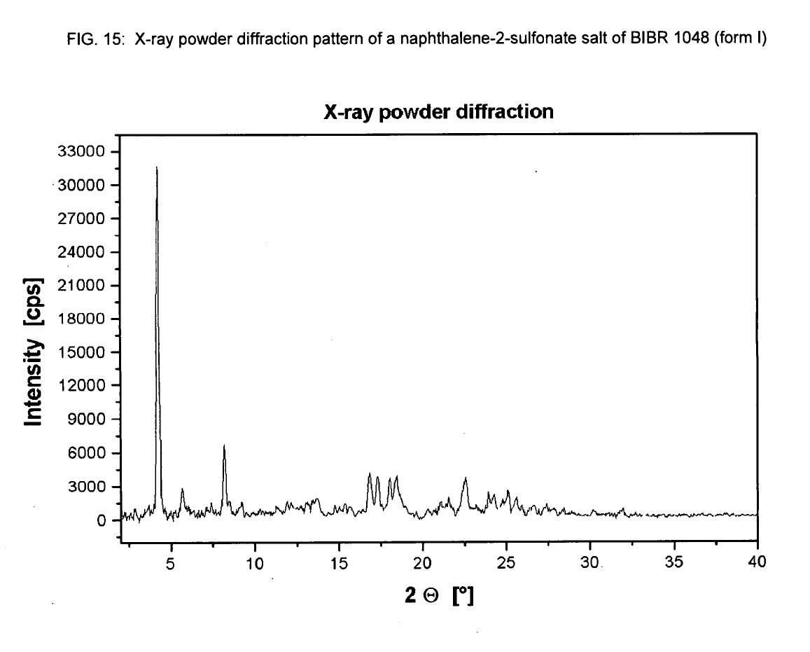

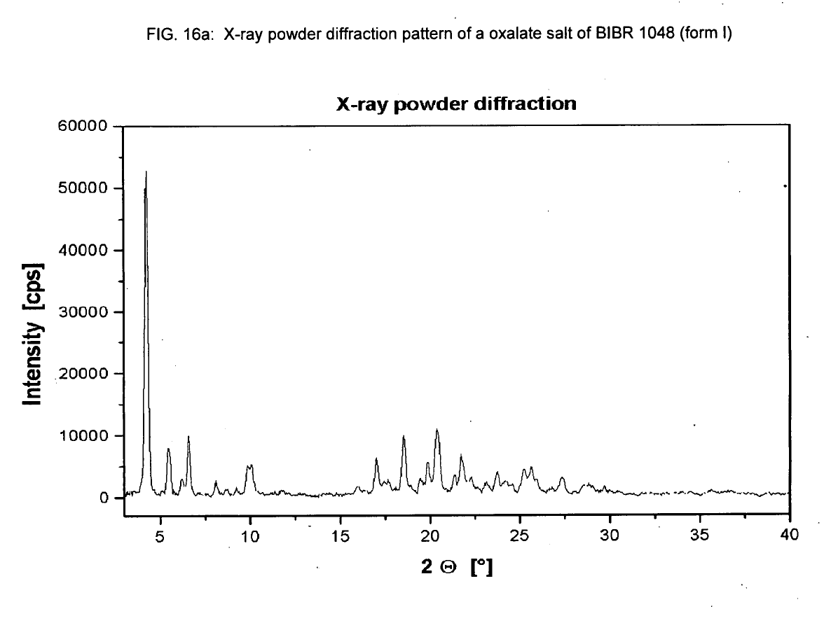

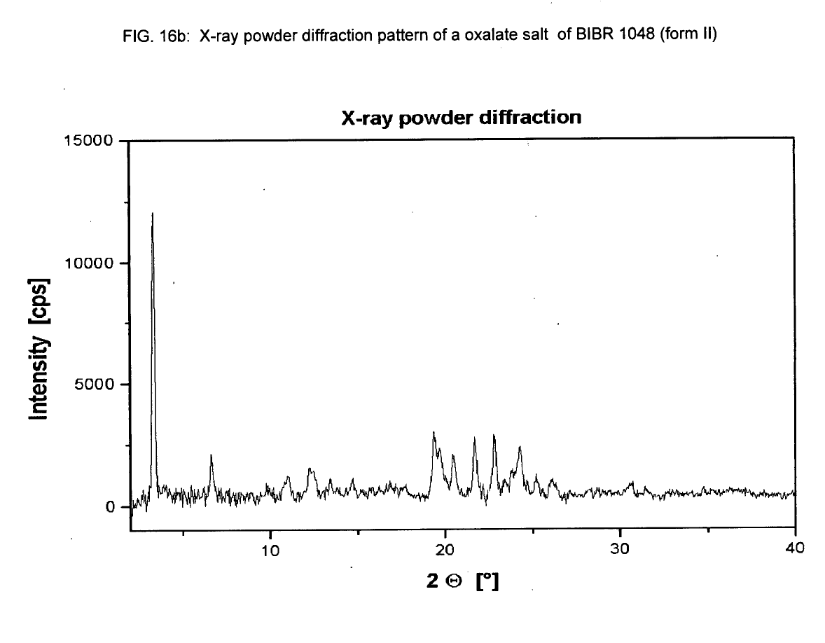

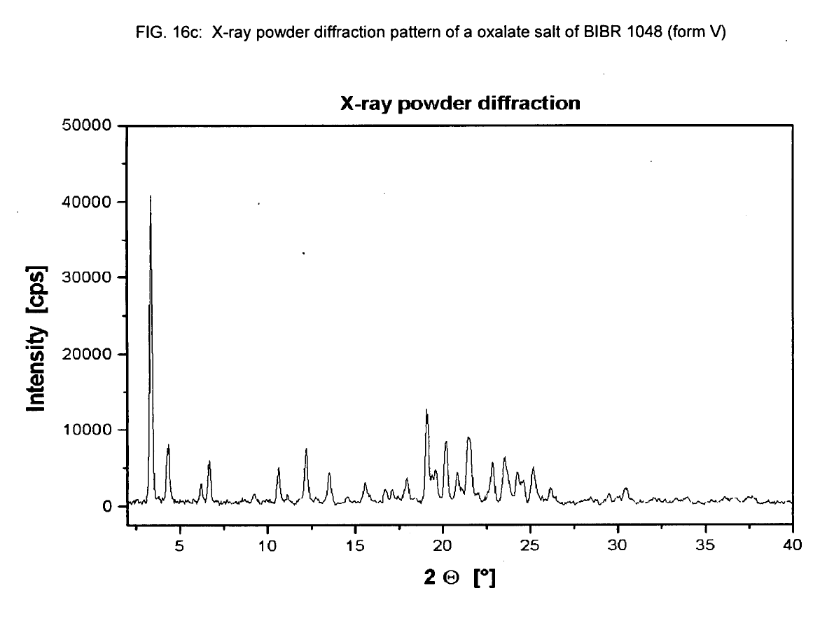

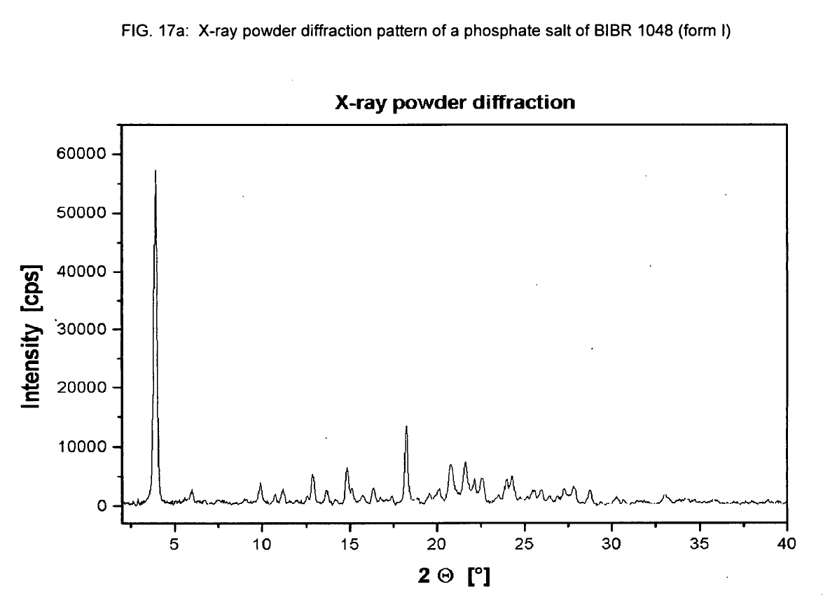

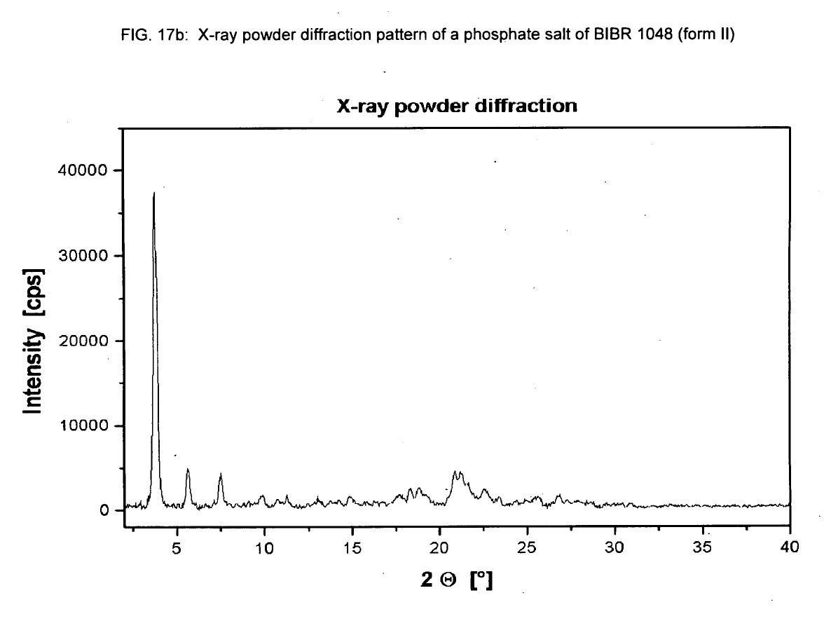

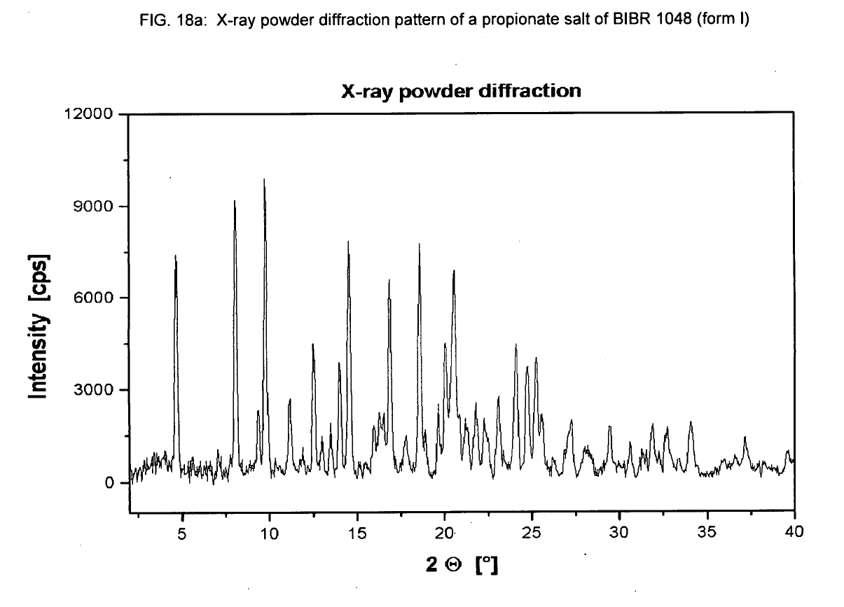

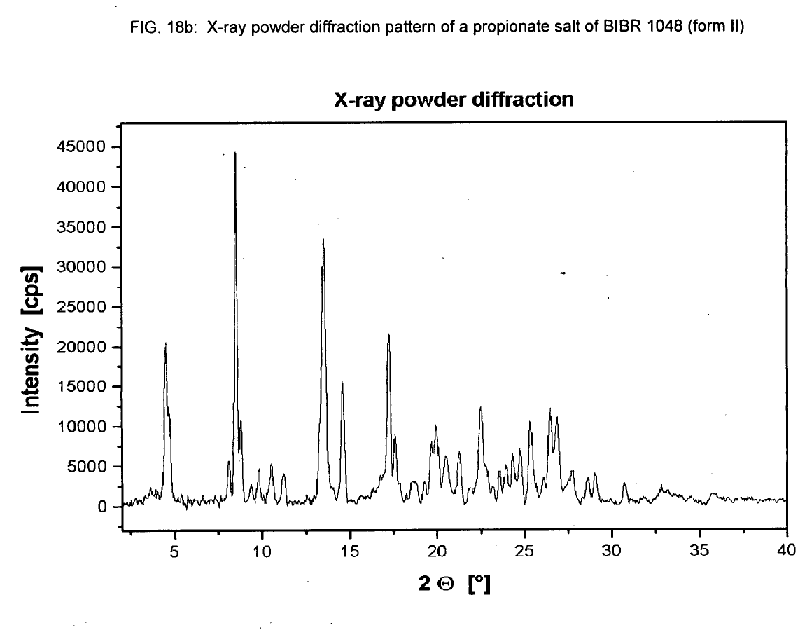

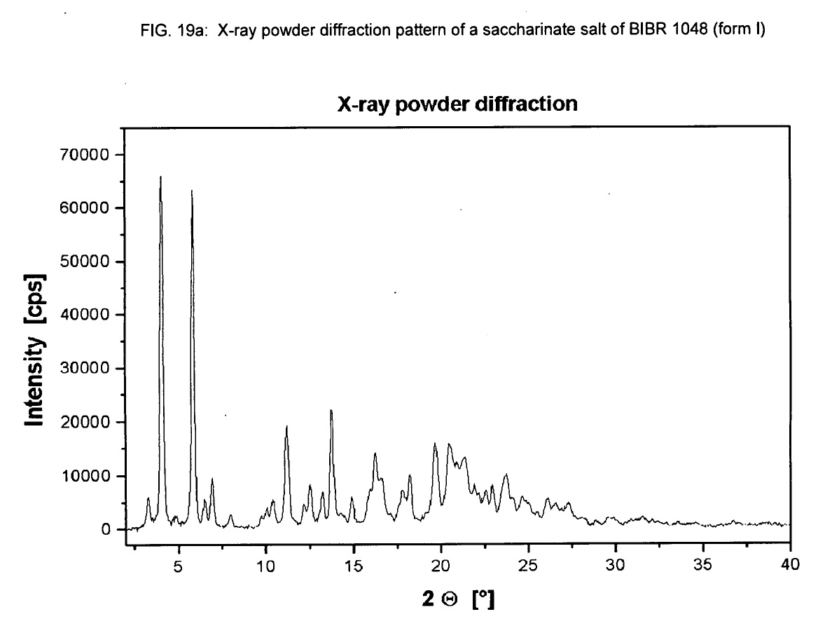

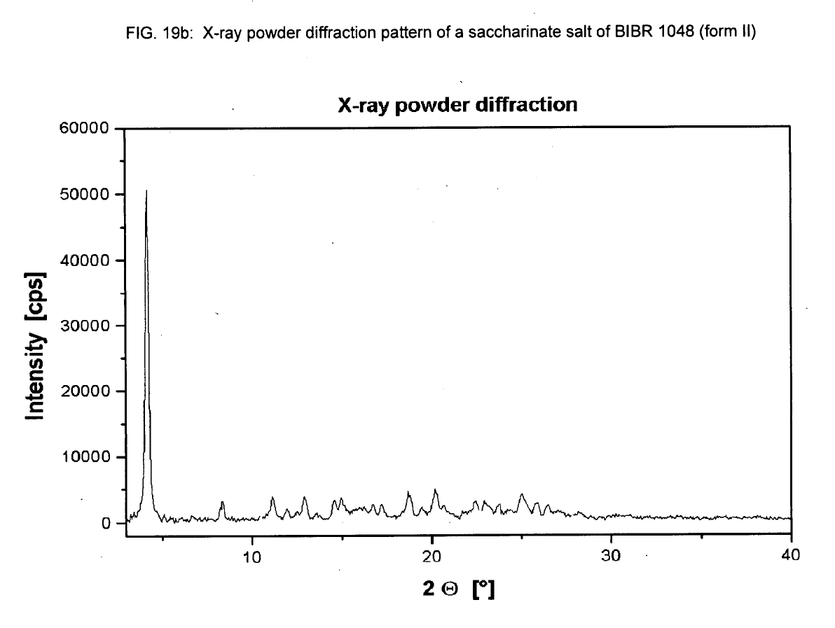

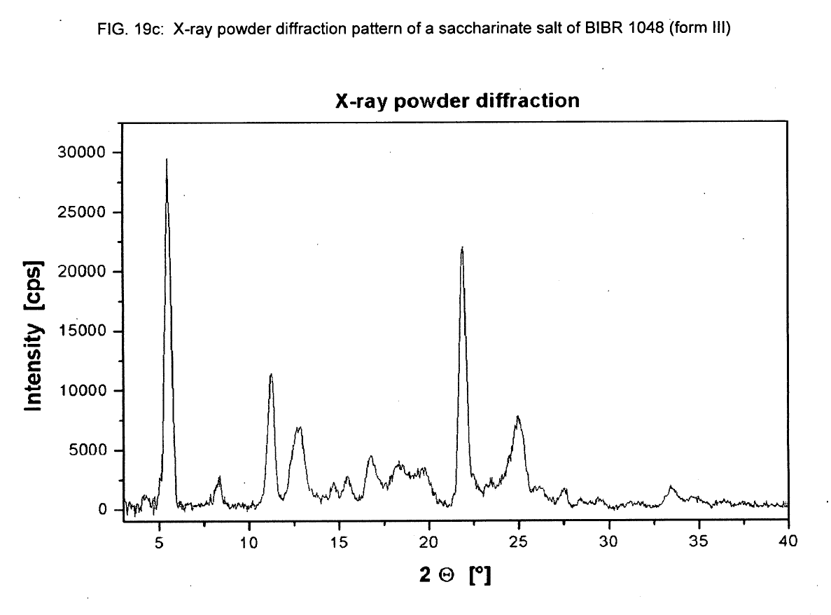

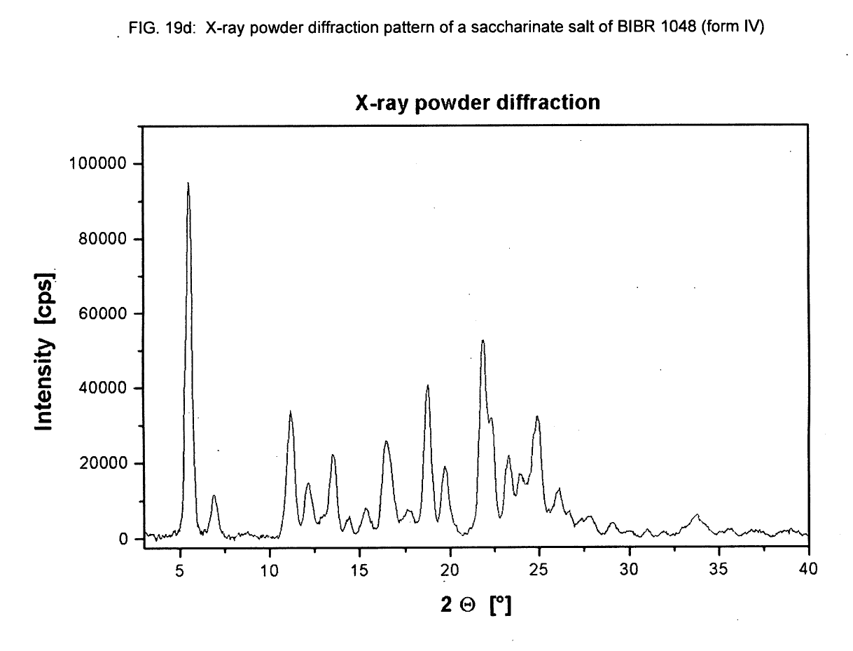

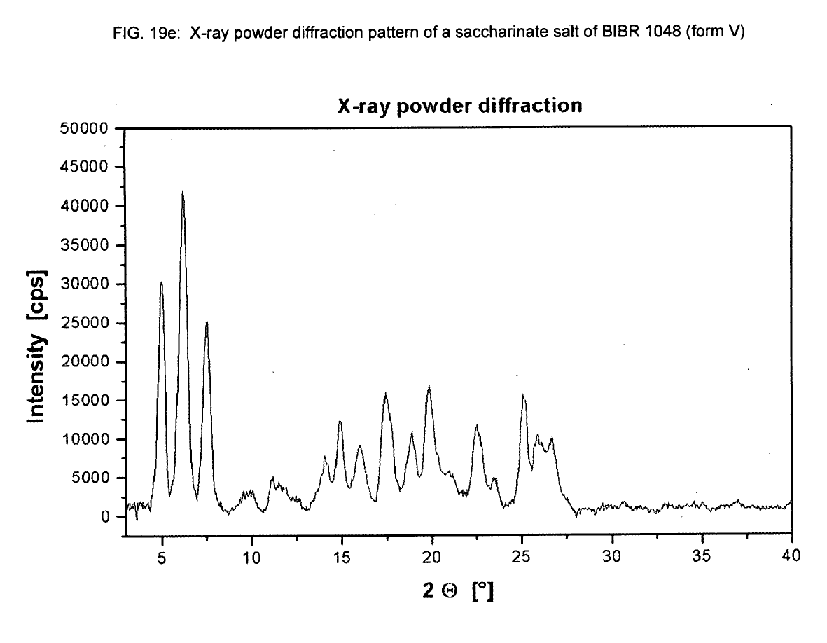

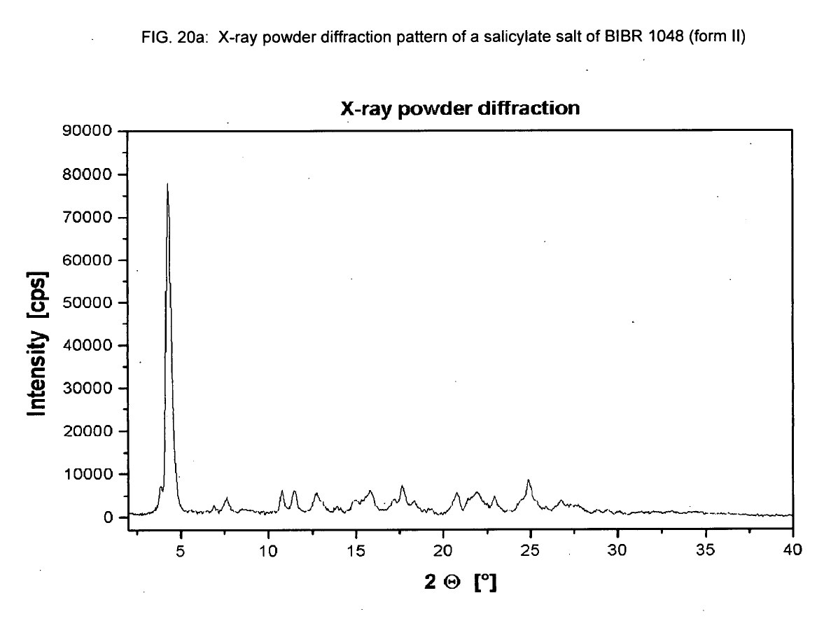

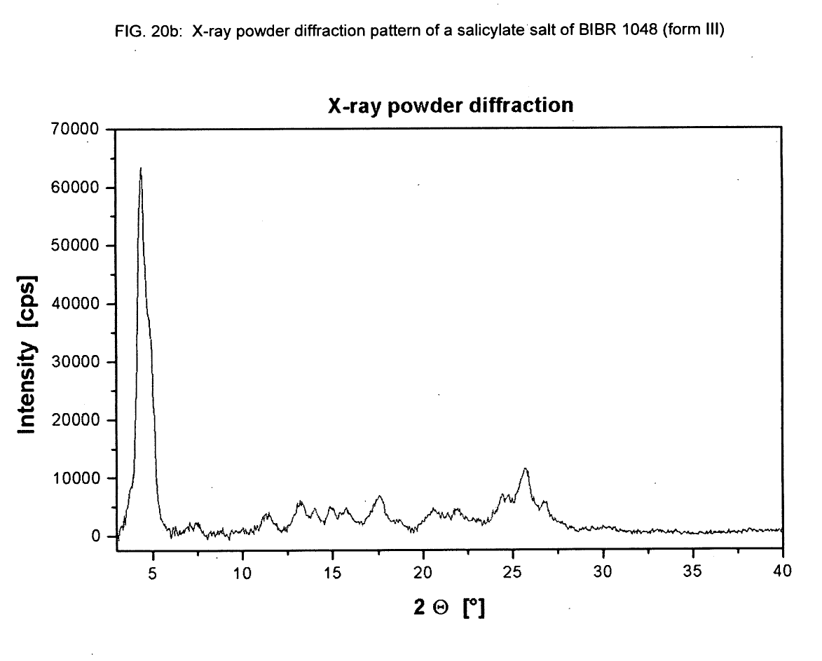

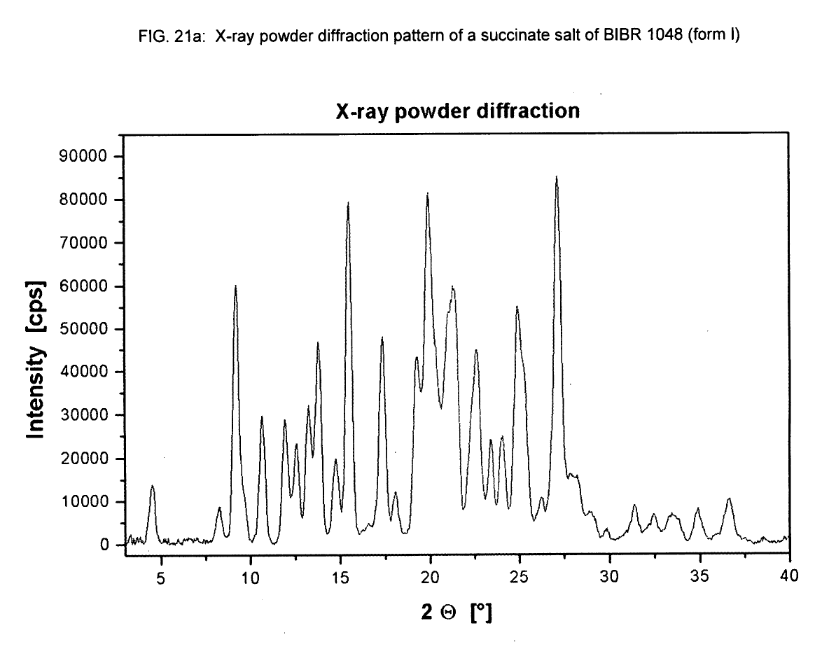

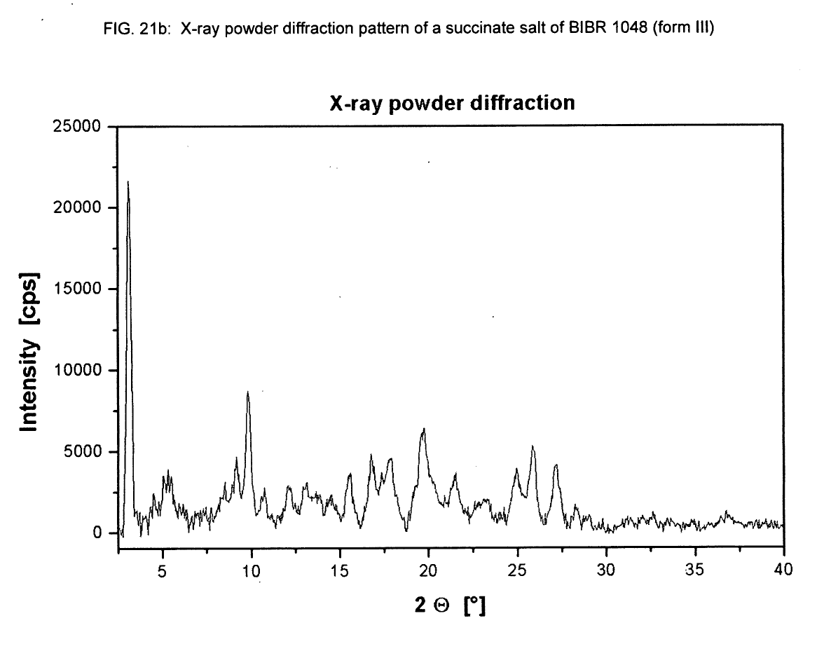

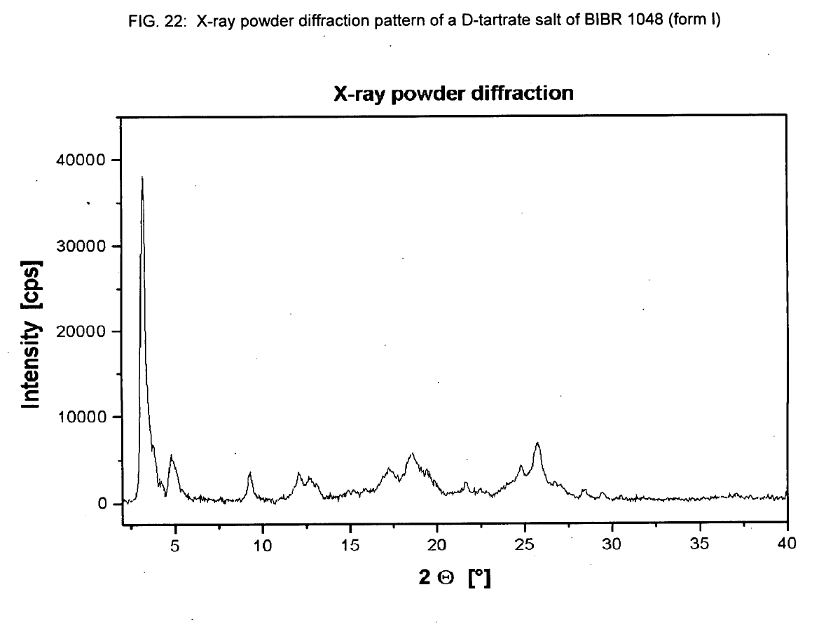

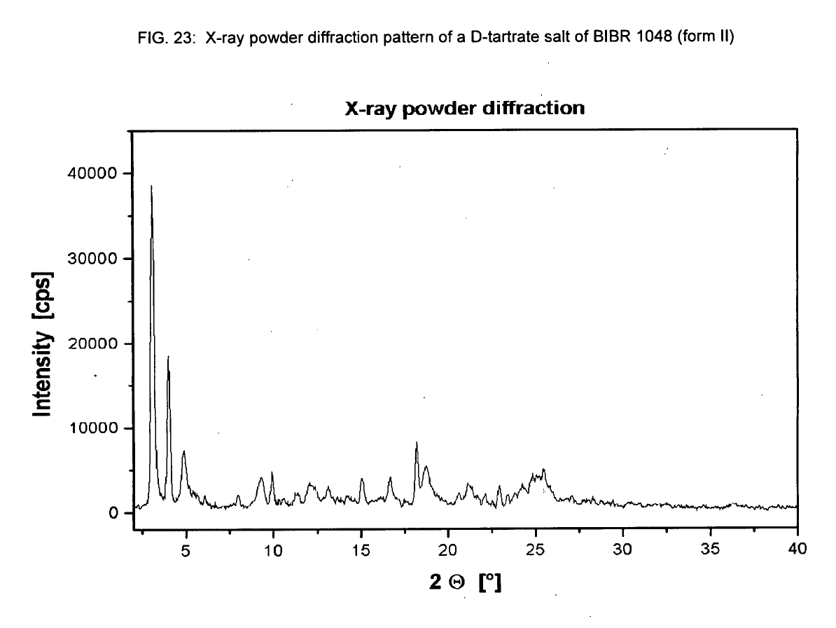

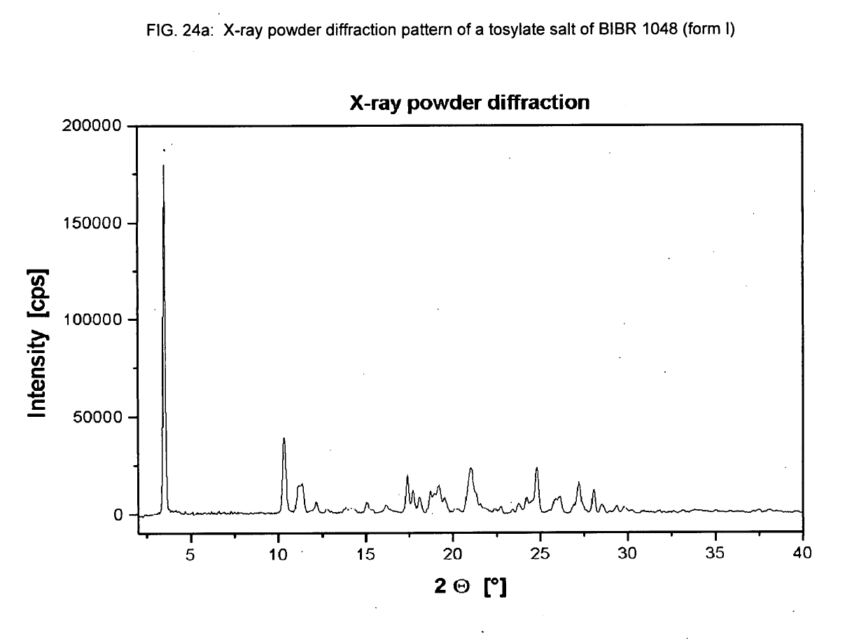

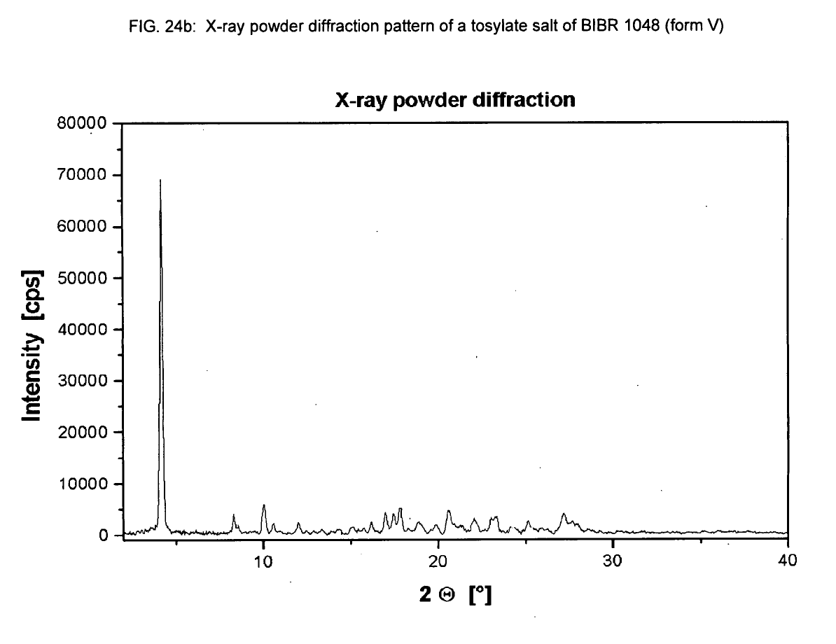

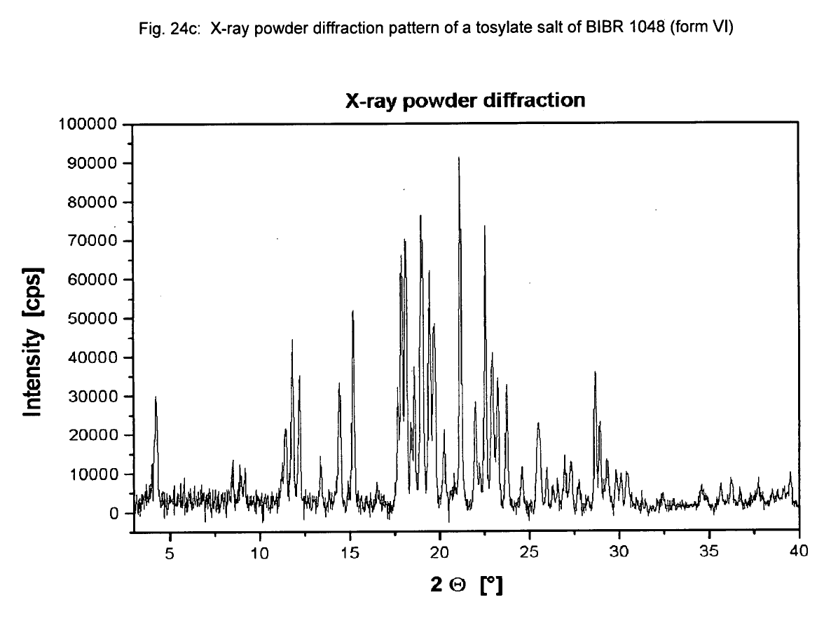

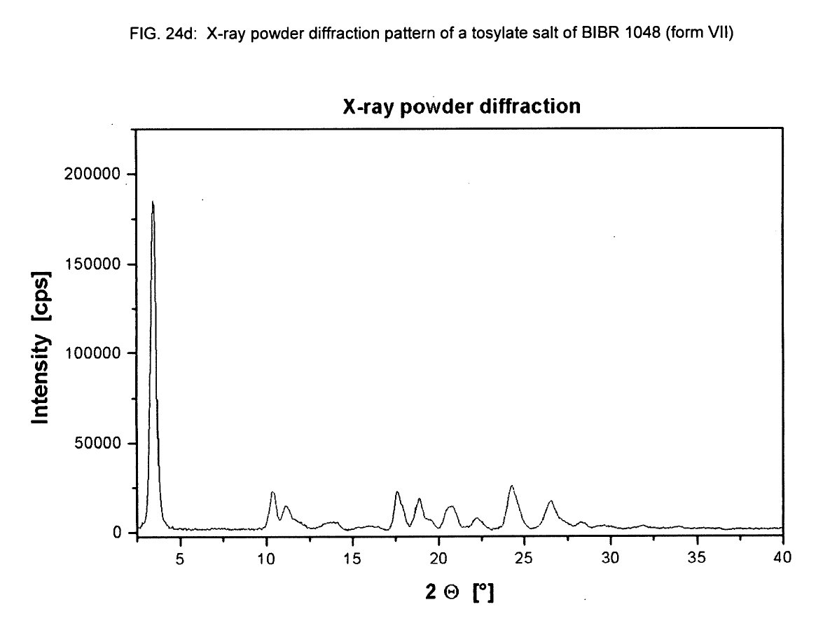

TABLE II
Thermal analysis and XRPD data of the different BIBR 1048 salts

| salt form | salt form | thermal analysis | |
|---|---|---|---|
| abbrev. | full name | (T_{fus} & LOD*) | XRPD - data |
| Gen2 | 2,5-dihydroxybenzoate | T_{fus} = 136° C. | see Tab. 1 & |
| | form II | LOD = 0.6% | FIG. 1 |
| Bes1 | besylate, form I | T_{fus} = 190° C. | see Tab. 2a & |
| | | LOD < 0.5% | FIG. 2a |
| Bes2 | besylate, form II | T_{fus} = 191° C. | see Tab. 2b & |
| | | LOD < 0.5% | FIG. 2b |
| Bes3 | besylate, form III | T_{fus} = 119° C. | see Tab. 2c & |
| | | LOD = 0.6% | FIG. 2c |
| HCl2 | chloride, form II | n.d. | see Tab. 3a & |
| | | | FIG. 3a |
| HCl5 | chloride, form V | n.d. | see Tab. 3b & |
| | | | FIG. 3b |
| HCl6 | chloride, form VI | n.d. | see Tab. 3c & |
| | | | FIG. 3c |
| Cyc1 | cyclamate, form I | T_{fus}: ca. 119° C. | see Tab. 4a & |
| | | LOD: n.d. | FIG. 4a |
| Cyc2 | cyclamate, form II | T_{fus}: ca. 110° C. | see Tab. 4b & |
| | | LOD: n.d. | FIG. 4b |
| Eds1 | edisylate, form I | T_{fus}: ca. 140° C. | see Tab. 5a & |
| | | LOD = 3.6% | FIG. 5a |
| Eds2 | edisylate, form II | T_{fus}: ca. 180° C. | see Tab. 5b & |
| | | LOD = 6.0% | FIG. 5b |
| Eds3 | edisylate, form III | T_{fus} = 121° C. | see Tab. 5c & |
| | | LOD < 0.5% | FIG. 5c |
| Eds4 | edisylate, form IV | T_{fus} = 130° C. | see Tab. 5d & |
| | | LOD: n.d. | FIG. 5d |
| Eds5 | edisylate, form V | T_{fus} = 116° C. | see Tab. 5e & |
| | | LOD = 2.4% | FIG. 5e |
| Ets1 | esylate, form I | T_{fus} = 203° C. | see Tab. 6 & |
| | | LOD = 0.5% | FIG. 6 |
| Fum3 | fumarate, form III | n.d. | see Tab. 7a & |
| | | | FIG. 7a |
| Fum4 | fumarate, form IV | T_{fus}: ca. 155° C. | see Tab. 7b & |
| | | LOD: n.d. | FIG. 7b |
| D-Glo1 | D-glucuronate, form I | T_{fus} = 156° C. | see Tab. 8 & |
| | | LOD = 0.4% | FIG. 8 |
| Gly1 | glycolate, form I | T_{fus} = 122° C. | see Tab. 9a & |
| | | LOD < 0.5% | FIG. 9a |
| Gly2 | glycolate, form II | T_{fus} = 92° C. | see Tab. 9b & |
| | | LOD = 1.6% | FIG. 9b |
| Gly3 | glycolate, form III | T_{fus} = 121° C. | see Tab. 9c & |
| | | LOD < 0.5% | FIG. 9c |
| Ise3 | isethionate, form III | T_{fus} = 139° C. | see Tab. 10 & |
| | | LOD = 0.5% | FIG. 10 |
| L-Mal1 | L-malate, form I | T_{fus} = 156° C. | see Tab. 11 & |
| | | LOD = 1.0% | FIG. 11 |
| D-Mal1 | D-malate, form I | T_{fus} = 158° C. | see Tab. 12 & |
| | | LOD: n.d. | FIG. 12 |
| Man1 | mandelate, form I | n.d. | see Tab. 13 & |
| | | | FIG. 13 |
| Nad1 | naphthalene-1,5- | T_{fus}: ca. 240° C. | see Tab. 14 & |
| | disulfonate, form I | LOD: n.d. | FIG. 14 |
| Nas1 | naphthalene-2- | n.d. | see Tab. 15 & |
| | sulfonate, form I | | FIG. 15 |
| Oxa1 | oxalate, form I | n.d. | see Tab. 16a & |
| | | | FIG. 16a |
| Oxa2 | oxalate, form II | n.d. | see Tab. 16b & |
| | | | FIG. 16b |
| Oxa5 | oxalate, form V | n.d. | see Tab. 16c & |
| | | | FIG. 16c |
| Pho1 | phosphate, form I | n.d. | see Tab. 17a & |
| | | | FIG. 17a |
| Pho2 | phosphate, form II | n.d. | see Tab. 17b & |
| | | | FIG. 17b |
| Pro1 | propionate, form I | n.d. | see Tab. 18a & |
| | | | FIG. 18a |
| Pro2 | propionate, form II | n.d. | see Tab. 18b & |
| | | | FIG. 18b |
| Sac1 | saccharinate, form I | T_{fus} = 142° C. | see Tab. 19a & |
| | | LOD = 0.2% | FIG. 19a |
| Sac2 | saccharinate, form II | T_{fus} = 137° C. | see Tab. 19b & |
| | | LOD = 0.5% | FIG. 19b |
| Sac3 | saccharinate, form III | T_{fus} = 142° C. | see Tab. 19c & |
| | | LOD < 0.5% | FIG. 19c |
| Sac4 | saccharinate, form IV | T_{fus} = 147° C. | see Tab. 19d & |
| | | LOD < 0.5% | FIG. 19d |
| Sac5 | saccharinate, form V | T_{fus} = 86° C. | see Tab. 19e & |
| | | LOD < 0.5% | FIG. 19e |
| Sal2 | salicylate, form II | T_{fus} = 152° C. | see Tab. 20a & |
| | | LOD = 0.5% | FIG. 20a |
| Sal3 | salicylate, form III | T_{fus} = 124° C. | see Tab. 20b & |
| | | LOD = 1.3% | FIG. 20b |
| Suc1 | succinate, form I | T_{fus} = 139° C. | see Tab. 21a & |
| | | LOD = 1.2% | FIG. 21a |
| Suc3 | succinate, form III | T_{fus} = 116° C. | see Tab. 21b & |
| | | LOD: n.d. | FIG. 21b |
| D-Tar1 | D-tartrate, form I | T_{fus} = 167° C. | see Tab. 22 & |
| | | LOD = 0.4% | FIG. 22 |
| D-Tar2 | D-tartrate, form II | n.d. | see Tab. 23 & |
| | | | FIG. 23 |
| Tos1 | tosylate, form I | T_{fus}: ca. 115° C. | see Tab. 24a & |
| | | LOD: n.d. | FIG. 24a |
| Tos5 | tosylate, form V | T_{fus}: ca. 155° C. | see Tab. 24b & |
| | | LOD: n.d. | FIG. 24b |
| Tos6 | tosylate, form VI | T_{fus}: ca. 153° C. | see Tab. 24c & |
| | | LOD = 0.1% | FIG. 24c |
| Tos7 | tosylate, form VII | T_{fus}: ca. 84° C. | see Tab. 24d & |
| | | LOD = 1.9% | FIG. 24d |

*LOD: loss on drying up to the melting point
n.d.: not determined

TABLE 1
X-ray powder reflections (up to 30° 2Θ) and intensities (normalized)
of a 2,5-dihydroxybenzoate salt of BIBR 1048 (form II)

| 2Θ [°] | d [Å] | I/Iₒ [%] |
|---|---|---|
| 3.86 | 22.87 | 8 |
| 4.29 | 20.61 | 100 |
| 6.91 | 12.80 | 4 |
| 7.64 | 11.58 | 4 |
| 8.54 | 10.35 | 3 |
| 10.27 | 8.61 | 6 |
| 10.70 | 8.27 | 9 |
| 11.46 | 7.72 | 12 |
| 12.71 | 6.97 | 11 |
| 12.96 | 6.83 | 6 |
| 13.86 | 6.39 | 3 |
| 14.90 | 5.95 | 6 |
| 15.26 | 5.81 | 5 |
| 15.70 | 5.65 | 7 |
| 17.08 | 5.19 | 8 |
| 17.61 | 5.04 | 18 |
| 18.36 | 4.83 | 5 |
| 19.15 | 4.63 | 5 |
| 20.06 | 4.43 | 2 |
| 20.74 | 4.28 | 13 |
| 21.50 | 4.13 | 6 |
| 21.86 | 4.07 | 15 |
| 22.24 | 4.00 | 8 |
| 22.88 | 3.89 | 10 |
| 24.31 | 3.66 | 7 |
| 24.82 | 3.59 | 19 |
| 25.10 | 3.55 | 11 |
| 25.73 | 3.46 | 7 |
| 26.57 | 3.35 | 10 |
| 27.18 | 3.28 | 5 |
| 27.59 | 3.23 | 7 |
| 27.86 | 3.20 | 4 |
| 28.66 | 3.11 | 2 |
| 29.26 | 3.05 | 3 |
| 29.90 | 2.99 | 3 |

TABLE 2a
X-ray powder reflections (up to 30° 2Θ) and intensities
(normalized) of a besylate salt of BIBR 1048 (form I)

| 2Θ [°] | d [Å] | I/Iₒ [%] |
|---|---|---|
| 4.29 | 20.59 | 100 |
| 8.36 | 10.57 | 5 |
| 8.60 | 10.28 | 7 |
| 9.70 | 9.11 | 4 |
| 10.02 | 8.82 | 11 |
| 11.17 | 7.91 | 3 |
| 11.64 | 7.60 | 3 |
| 12.00 | 7.37 | 8 |
| 12.35 | 7.16 | 3 |
| 13.36 | 6.62 | 3 |
| 14.95 | 5.92 | 2 |
| 15.60 | 5.67 | 7 |
| 16.17 | 5.48 | 2 |
| 16.67 | 5.31 | 6 |
| 17.55 | 5.05 | 0 |
| 18.32 | 4.84 | 18 |
| 18.68 | 4.75 | 10 |
| 19.37 | 4.58 | 8 |
| 20.14 | 4.41 | 7 |
| 20.40 | 4.35 | 11 |
| 20.94 | 4.24 | 2 |
| 21.33 | 4.16 | 1 |
| 21.87 | 4.06 | 4 |
| 22.15 | 4.01 | 6 |
| 22.72 | 3.91 | 5 |
| 23.00 | 3.86 | 9 |
| 24.14 | 3.68 | 6 |
| 24.43 | 3.64 | 4 |
| 24.92 | 3.57 | 2 |
| 25.19 | 3.53 | 1 |
| 25.83 | 3.45 | 2 |
| 26.67 | 3.34 | 4 |
| 26.92 | 3.31 | 3 |
| 27.43 | 3.25 | 8 |
| 27.96 | 3.19 | 3 |
| 28.19 | 3.16 | 3 |
| 29.16 | 3.06 | 1 |
| 29.48 | 3.03 | 1 |
| 30.35 | 2.94 | 1 |

TABLE 2b
X-ray powder reflections (up to 30° 2Θ) and intensities (normalized)
of a besylate salt of BIBR 1048 (form II)

| 2Θ [°] | d [Å] | I/Iₒ [%] |
|---|---|---|
| 4.26 | 20.72 | 100 |
| 8.34 | 10.60 | 9 |
| 8.54 | 10.34 | 9 |
| 10.55 | 8.38 | 20 |
| 11.66 | 7.59 | 2 |
| 12.04 | 7.34 | 3 |
| 12.58 | 7.03 | 5 |
| 13.45 | 6.58 | 3 |
| 14.31 | 6.18 | 1 |
| 14.59 | 6.07 | 2 |
| 15.08 | 5.87 | 5 |
| 16.03 | 5.52 | 2 |
| 16.69 | 5.31 | 13 |
| 17.28 | 5.13 | 3 |
| 17.86 | 4.96 | 10 |
| 18.17 | 4.88 | 4 |
| 18.71 | 4.74 | 9 |
| 19.05 | 4.66 | 13 |
| 19.52 | 4.54 | 13 |
| 19.92 | 4.45 | 11 |
| 20.60 | 4.31 | 25 |
| 21.02 | 4.22 | 3 |
| 21.83 | 4.07 | 7 |
| 22.20 | 4.00 | 4 |
| 22.67 | 3.92 | 8 |
| 23.19 | 3.83 | 8 |
| 23.88 | 3.72 | 9 |
| 24.20 | 3.67 | 4 |
| 24.65 | 3.61 | 5 |
| 25.20 | 3.53 | 3 |
| 25.72 | 3.46 | 4 |
| 26.51 | 3.36 | 2 |
| 26.89 | 3.31 | 5 |
| 27.51 | 3.24 | 11 |
| 27.83 | 3.20 | 14 |
| 28.82 | 3.09 | 2 |
| 29.49 | 3.03 | 2 |

TABLE 2c
X-ray powder reflections (up to 30° 2Θ) and intensities (normalized)
of a besylate salt of BIBR 1048 (form III)

| 2Θ [°] | d [Å] | I/Iₒ [%] |
|---|---|---|
| 4.99 | 17.69 | 13 |
| 5.55 | 15.90 | 59 |
| 7.87 | 11.22 | 25 |
| 9.89 | 8.94 | 25 |
| 11.00 | 8.03 | 19 |
| 12.15 | 7.28 | 43 |
| 13.49 | 6.56 | 26 |
| 14.02 | 6.31 | 8 |
| 15.11 | 5.86 | 36 |
| 15.67 | 5.65 | 16 |
| 16.29 | 5.44 | 16 |
| 16.80 | 5.27 | 15 |
| 17.18 | 5.16 | 29 |
| 18.47 | 4.80 | 100 |
| 19.11 | 4.64 | 14 |
| 19.86 | 4.47 | 22 |
| 20.40 | 4.35 | 35 |
| 20.87 | 4.25 | 28 |
| 21.97 | 4.04 | 35 |
| 23.68 | 3.75 | 52 |
| 24.63 | 3.61 | 76 |
| 25.47 | 3.49 | 12 |
| 26.23 | 3.39 | 22 |
| 27.35 | 3.26 | 12 |
| 28.60 | 3.12 | 6 |
| 30.56 | 2.92 | 10 |

TABLE 3a
X-ray powder reflections (up to 30° 2Θ) and intensities (normalized)
of a chloride salt of BIBR 1048 (form II)

| 2Θ [°] | d [Å] | I/Iₒ [%] |
|---|---|---|
| 3.63 | 24.36 | 15 |
| 4.42 | 19.98 | 46 |
| 5.08 | 17.38 | 102 |
| 7.19 | 12.29 | 12 |
| 9.51 | 9.30 | 14 |
| 10.28 | 8.60 | 20 |
| 10.96 | 8.07 | 15 |
| 11.38 | 7.77 | 8 |
| 12.19 | 7.26 | 14 |
| 12.59 | 7.03 | 11 |
| 13.31 | 6.65 | 12 |
| 13.74 | 6.44 | 3 |
| 14.84 | 5.97 | 12 |
| 15.30 | 5.79 | 15 |
| 15.90 | 5.57 | 5 |
| 16.73 | 5.30 | 13 |
| 17.01 | 5.21 | 14 |
| 17.39 | 5.10 | 14 |
| 17.90 | 4.95 | 15 |
| 18.18 | 4.88 | 16 |
| 18.68 | 4.75 | 19 |
| 19.44 | 4.57 | 18 |
| 19.86 | 4.47 | 11 |
| 20.26 | 4.38 | 14 |
| 20.95 | 4.24 | 16 |
| 21.32 | 4.17 | 16 |
| 21.66 | 4.10 | 12 |
| 22.13 | 4.02 | 18 |
| 22.59 | 3.94 | 16 |
| 22.98 | 3.87 | 14 |
| 23.46 | 3.79 | 20 |
| 24.02 | 3.70 | 13 |
| 24.66 | 3.61 | 15 |
| 25.07 | 3.55 | 22 |
| 25.30 | 3.52 | 17 |
| 26.12 | 3.41 | 16 |
| 27.25 | 3.27 | 11 |
| 28.10 | 3.18 | 6 |
| 29.30 | 3.05 | 7 |

TABLE 3b
X-ray powder reflections (up to 30° 2Θ) and intensities (normalized)
of a chloride salt of BIBR 1048 (form V)

| 2Θ [°] | d [Å] | I/Iₒ [%] |
|---|---|---|
| 3.87 | 22.80 | 100 |
| 6.49 | 13.62 | 28 |
| 7.81 | 11.32 | 5 |
| 9.95 | 8.89 | 26 |
| 10.50 | 8.42 | 31 |
| 10.97 | 8.06 | 22 |
| 11.24 | 7.87 | 19 |
| 11.65 | 7.60 | 21 |
| 11.98 | 7.39 | 32 |
| 12.71 | 6.97 | 38 |
| 13.21 | 6.70 | 24 |
| 13.84 | 6.40 | 10 |
| 14.27 | 6.20 | 32 |
| 15.35 | 5.77 | 31 |
| 16.73 | 5.30 | 26 |
| 17.40 | 5.10 | 19 |
| 17.70 | 5.01 | 18 |
| 18.42 | 4.82 | 19 |
| 19.11 | 4.64 | 25 |
| 19.45 | 4.56 | 32 |
| 19.78 | 4.49 | 26 |
| 19.98 | 4.44 | 32 |
| 20.62 | 4.31 | 10 |
| 21.17 | 4.20 | 50 |
| 21.57 | 4.12 | 37 |
| 21.82 | 4.07 | 30 |
| 22.18 | 4.01 | 20 |
| 22.49 | 3.95 | 35 |
| 22.78 | 3.90 | 15 |
| 23.40 | 3.80 | 31 |
| 23.74 | 3.75 | 15 |
| 24.45 | 3.64 | 36 |
| 24.85 | 3.58 | 25 |
| 25.50 | 3.49 | 12 |
| 25.90 | 3.44 | 28 |
| 26.22 | 3.40 | 23 |
| 26.73 | 3.34 | 17 |
| 27.41 | 3.25 | 16 |
| 28.01 | 3.18 | 14 |
| 28.83 | 3.10 | 17 |
| 29.50 | 3.03 | 17 |

TABLE 3c
X-ray powder reflections (up to 30° 2Θ) and intensities (normalized)
of a chloride salt of BIBR 1048 (form VI)

| 2Θ [°] | d [Å] | I/Iₒ [%] |
|---|---|---|
| 3.62 | 24.43 | 100 |
| 3.82 | 23.13 | 20 |
| 7.19 | 12.29 | 22 |
| 9.53 | 9.28 | 51 |
| 9.78 | 9.04 | 25 |
| 10.58 | 8.36 | 10 |
| 10.83 | 8.17 | 24 |
| 11.02 | 8.03 | 17 |
| 11.24 | 7.87 | 28 |
| 11.66 | 7.59 | 5 |
| 12.21 | 7.25 | 40 |
| 12.63 | 7.01 | 23 |
| 13.43 | 6.59 | 10 |
| 14.32 | 6.19 | 5 |
| 14.54 | 6.09 | 3 |
| 15.29 | 5.79 | 25 |
| 15.84 | 5.59 | 20 |
| 16.22 | 5.46 | 4 |
| 16.74 | 5.30 | 7 |
| 17.37 | 5.11 | 34 |
| 18.27 | 4.86 | 18 |
| 18.81 | 4.72 | 38 |
| 19.10 | 4.65 | 20 |
| 19.44 | 4.57 | 59 |
| 20.90 | 4.25 | 31 |
| 21.33 | 4.16 | 45 |
| 22.19 | 4.01 | 54 |
| 22.62 | 3.93 | 36 |
| 23.03 | 3.86 | 29 |
| 23.47 | 3.79 | 66 |
| 24.05 | 3.70 | 26 |
| 24.75 | 3.60 | 25 |
| 25.10 | 3.55 | 15 |
| 26.07 | 3.42 | 24 |
| 26.71 | 3.34 | 17 |
| 27.30 | 3.27 | 26 |
| 27.66 | 3.22 | 14 |
| 28.29 | 3.15 | 21 |
| 28.28 | 3.16 | 9 |
| 29.52 | 3.03 | 16 |
| 30.03 | 2.98 | 14 |

TABLE 4a
X-ray powder reflections (up to 30° 2Θ) and intensities (normalized)
of a cyclamate salt of BIBR 1048 (form I)

| 2Θ [°] | d [Å] | I/Iₒ [%] |
|---|---|---|
| 3.40 | 26.00 | 100 |
| 4.55 | 19.41 | 12 |
| 5.22 | 16.93 | 2 |
| 6.71 | 13.18 | 4 |
| 7.07 | 12.50 | 5 |
| 9.05 | 9.77 | 4 |
| 10.70 | 8.27 | 20 |
| 11.56 | 7.66 | 6 |
| 12.46 | 7.10 | 4 |
| 13.50 | 6.56 | 6 |
| 15.58 | 5.69 | 5 |
| 16.94 | 5.23 | 4 |
| 17.34 | 5.11 | 4 |
| 18.03 | 4.92 | 11 |
| 18.42 | 4.82 | 10 |
| 19.28 | 4.60 | 7 |
| 20.03 | 4.43 | 8 |
| 21.15 | 4.20 | 9 |
| 21.65 | 4.11 | 7 |
| 22.34 | 3.98 | 4 |
| 22.67 | 3.92 | 5 |
| 23.58 | 3.77 | 5 |
| 24.10 | 3.69 | 2 |
| 24.42 | 3.65 | 2 |
| 24.88 | 3.58 | 6 |
| 25.86 | 3.45 | 6 |
| 26.90 | 3.31 | 7 |
| 27.38 | 3.26 | 2 |
| 27.86 | 3.20 | 2 |
| 28.32 | 3.15 | 4 |
| 29.03 | 3.08 | 5 |

TABLE 4b
X-ray powder reflections (up to 30° 2Θ) and intensities (normalized)
of a cyclamate salt of BIBR 1048 (form II)

| 2Θ [°] | d [Å] | I/Iₒ [%] |
|---|---|---|
| 3.41 | 25.93 | 10 |
| 3.78 | 23.36 | 100 |
| 4.90 | 18.03 | 1 |
| 7.53 | 11.75 | 4 |
| 8.84 | 10.01 | 5 |
| 9.26 | 9.55 | 7 |
| 9.52 | 9.29 | 7 |
| 9.97 | 8.87 | 9 |
| 10.57 | 8.37 | 6 |
| 11.28 | 7.84 | 13 |
| 11.58 | 7.64 | 3 |
| 12.90 | 6.86 | 2 |
| 13.26 | 6.68 | 3 |
| 13.78 | 6.43 | 3 |
| 15.02 | 5.90 | 3 |
| 15.48 | 5.72 | 6 |
| 15.92 | 5.57 | 6 |
| 16.60 | 5.34 | 6 |
| 17.22 | 5.15 | 6 |
| 17.34 | 5.11 | 7 |
| 18.06 | 4.91 | 2 |
| 18.40 | 4.82 | 11 |
| 19.28 | 4.60 | 6 |
| 20.18 | 4.40 | 8 |
| 20.89 | 4.25 | 6 |
| 21.65 | 4.10 | 7 |
| 22.52 | 3.95 | 5 |
| 22.83 | 3.90 | 6 |
| 23.42 | 3.80 | 3 |
| 24.82 | 3.59 | 13 |
| 25.74 | 3.46 | 6 |
| 26.38 | 3.38 | 3 |
| 26.74 | 3.33 | 4 |
| 27.49 | 3.24 | 10 |
| 27.82 | 3.21 | 3 |
| 28.54 | 3.13 | 2 |

TABLE 5a
X-ray powder reflections (up to 30° 2Θ) and intensities (normalized)
of a edysilate salt of BIBR 1048 (form I)

| 2Θ [°] | d [Å] | I/Iₒ [%] |
|---|---|---|
| 3.07 | 28.76 | 79 |
| 3.93 | 22.46 | 100 |
| 7.85 | 11.26 | 7 |
| 8.11 | 10.90 | 4 |
| 8.44 | 10.48 | 3 |
| 9.22 | 9.59 | 3 |
| 10.20 | 8.67 | 15 |
| 10.98 | 8.06 | 12 |
| 11.50 | 7.69 | 16 |
| 11.85 | 7.47 | 12 |
| 12.43 | 7.12 | 54 |
| 13.17 | 6.72 | 34 |
| 13.50 | 6.56 | 15 |
| 14.13 | 6.27 | 23 |
| 15.90 | 5.57 | 3 |
| 16.30 | 5.44 | 3 |
| 17.16 | 5.17 | 31 |
| 17.53 | 5.06 | 12 |
| 18.19 | 4.88 | 27 |
| 18.70 | 4.74 | 51 |
| 19.12 | 4.64 | 40 |
| 19.66 | 4.52 | 5 |
| 20.32 | 4.37 | 17 |
| 20.70 | 4.29 | 12 |
| 21.79 | 4.08 | 47 |
| 22.28 | 3.99 | 28 |
| 22.69 | 3.92 | 26 |
| 23.24 | 3.83 | 50 |
| 23.74 | 3.75 | 14 |
| 23.92 | 3.72 | 22 |
| 24.31 | 3.66 | 13 |
| 24.70 | 3.60 | 12 |
| 25.05 | 3.55 | 10 |
| 25.66 | 3.47 | 10 |
| 26.20 | 3.40 | 8 |
| 26.53 | 3.36 | 11 |
| 26.66 | 3.34 | 9 |
| 27.02 | 3.30 | 4 |
| 27.22 | 3.28 | 4 |
| 27.78 | 3.21 | 6 |
| 28.03 | 3.18 | 19 |
| 29.34 | 3.04 | 13 |
| 29.61 | 3.02 | 18 |

TABLE 5b
X-ray powder reflections (up to 30° 2Θ) and intensities (normalized)
of a edysilate salt of BIBR 1048 (form II)

| 2Θ [°] | d [Å] | I/Iₒ [%] |
|---|---|---|
| 3.08 | 28.65 | 11 |
| 3.97 | 22.28 | 99 |
| 4.56 | 19.39 | 16 |
| 6.54 | 13.51 | 5 |
| 7.90 | 11.19 | 8 |
| 10.13 | 8.73 | 14 |
| 10.96 | 8.07 | 17 |
| 12.16 | 7.28 | 6 |
| 14.16 | 6.25 | 12 |
| 15.91 | 5.57 | 11 |
| 17.02 | 5.21 | 17 |
| 17.66 | 5.02 | 5 |
| 18.14 | 4.89 | 6 |
| 18.45 | 4.81 | 8 |
| 19.16 | 4.63 | 9 |
| 19.72 | 4.50 | 9 |
| 20.34 | 4.37 | 14 |
| 20.62 | 4.31 | 10 |
| 21.42 | 4.15 | 13 |
| 21.78 | 4.08 | 13 |
| 22.10 | 4.02 | 10 |
| 22.37 | 3.97 | 14 |
| 22.66 | 3.92 | 10 |
| 22.99 | 3.87 | 14 |
| 23.26 | 3.82 | 9 |
| 23.62 | 3.77 | 8 |
| 23.91 | 3.72 | 8 |
| 24.47 | 3.64 | 6 |
| 24.70 | 3.60 | 8 |
| 25.73 | 3.46 | 10 |
| 26.22 | 3.40 | 5 |
| 27.50 | 3.24 | 3 |
| 28.02 | 3.18 | 3 |
| 29.54 | 3.02 | 4 |

TABLE 5c
X-ray powder reflections (up to 30° 2Θ) and intensities (normalized)
of a edysilate salt of BIBR 1048 (form III)

| 2Θ [°] | d [Å] | I/Iₒ [%] |
|---|---|---|
| 5.25 | 16.81 | 24 |
| 6.01 | 14.69 | 13 |
| 8.00 | 11.04 | 8 |
| 9.11 | 9.69 | 10 |
| 10.03 | 8.82 | 3 |
| 10.59 | 8.35 | 24 |
| 12.13 | 7.29 | 22 |
| 12.75 | 6.94 | 23 |
| 13.24 | 6.68 | 27 |
| 14.49 | 6.11 | 12 |
| 15.16 | 5.84 | 6 |
| 16.40 | 5.40 | 23 |
| 16.97 | 5.22 | 17 |
| 17.46 | 5.07 | 59 |
| 18.08 | 4.90 | 29 |
| 19.44 | 4.56 | 31 |
| 20.28 | 4.37 | 27 |
| 21.69 | 4.09 | 31 |
| 22.41 | 3.96 | 100 |
| 23.11 | 3.85 | 27 |
| 23.85 | 3.73 | 20 |
| 24.45 | 3.64 | 12 |
| 24.73 | 3.60 | 8 |
| 25.66 | 3.47 | 19 |
| 26.43 | 3.37 | 15 |
| 26.79 | 3.32 | 18 |
| 28.33 | 3.15 | 5 |
| 28.62 | 3.12 | 8 |
| 29.05 | 3.07 | 5 |
| 29.25 | 3.05 | 5 |
| 29.72 | 3.00 | 7 |
| 29.96 | 2.98 | 7 |

TABLE 5d
X-ray powder reflections (up to 30° 2Θ) and intensities (normalized)
of a edysilate salt of BIBR 1048 (form IV)

| 2Θ [°] | d [Å] | I/Iₒ [%] |
|---|---|---|
| 4.29 | 20.60 | 100 |
| 7.33 | 12.05 | 4 |
| 8.25 | 10.71 | 14 |
| 8.52 | 10.37 | 29 |
| 9.56 | 9.24 | 21 |
| 11.09 | 7.97 | 23 |
| 11.85 | 7.46 | 7 |
| 12.39 | 7.14 | 22 |
| 12.82 | 6.90 | 29 |
| 13.33 | 6.64 | 5 |
| 14.37 | 6.16 | 19 |
| 14.84 | 5.97 | 5 |
| 15.56 | 5.69 | 14 |
| 17.06 | 5.19 | 16 |
| 17.95 | 4.94 | 45 |
| 18.56 | 4.78 | 26 |
| 19.00 | 4.67 | 14 |
| 20.38 | 4.35 | 50 |
| 21.45 | 4.14 | 50 |
| 22.34 | 3.98 | 41 |
| 22.69 | 3.92 | 27 |
| 23.21 | 3.83 | 19 |
| 23.90 | 3.72 | 14 |
| 25.17 | 3.54 | 19 |
| 25.81 | 3.45 | 18 |
| 26.48 | 3.36 | 18 |
| 26.92 | 3.31 | 7 |
| 27.72 | 3.22 | 6 |
| 27.72 | 3.22 | 6 |
| 28.23 | 3.16 | 5 |
| 28.94 | 3.08 | 8 |
| 29.35 | 3.04 | 8 |

TABLE 5e
X-ray powder reflections (up to 30° 2Θ) and intensities (normalized)
of a edysilate salt of BIBR 1048 (form V)

| 2Θ [°] | d [Å] | I/Iₒ [%] |
|---|---|---|
| 3.94 | 22.42 | 25 |
| 5.63 | 15.69 | 8 |
| 7.79 | 11.34 | 2 |
| 8.38 | 10.54 | 2 |
| 10.32 | 8.57 | 11 |
| 11.10 | 7.97 | 19 |
| 12.90 | 6.86 | 30 |
| 14.03 | 6.31 | 21 |
| 15.70 | 5.64 | 10 |
| 16.38 | 5.41 | 7 |
| 16.80 | 5.27 | 19 |
| 17.17 | 5.16 | 12 |
| 17.70 | 5.01 | 25 |
| 18.56 | 4.78 | 9 |
| 19.04 | 4.66 | 25 |
| 20.02 | 4.43 | 100 |
| 20.91 | 4.24 | 23 |
| 21.28 | 4.17 | 23 |
| 22.39 | 3.97 | 20 |
| 23.53 | 3.78 | 11 |
| 24.66 | 3.61 | 9 |
| 25.59 | 3.48 | 21 |
| 25.95 | 3.43 | 33 |
| 26.98 | 3.30 | 3 |
| 28.31 | 3.15 | 6 |
| 28.61 | 3.12 | 6 |
| 29.25 | 3.05 | 12 |

TABLE 6
X-ray powder reflections (up to 30° 2Θ) and intensities (normalized) of
a esylate salt of BIBR 1048 (form I)

| 2Θ [°] | d [Å] | I/Iₒ [%] |
|---|---|---|
| 4.35 | 20.30 | 34 |
| 8.87 | 9.97 | 11 |
| 9.57 | 9.24 | 21 |
| 9.83 | 9.00 | 23 |
| 11.12 | 7.96 | 22 |
| 12.22 | 7.24 | 35 |
| 13.46 | 6.58 | 16 |
| 13.83 | 6.40 | 23 |
| 14.94 | 5.93 | 37 |
| 15.82 | 5.60 | 6 |
| 16.36 | 5.42 | 10 |
| 17.07 | 5.20 | 9 |
| 17.76 | 4.99 | 89 |
| 18.42 | 4.82 | 42 |
| 19.11 | 4.64 | 59 |
| 19.70 | 4.51 | 21 |
| 19.99 | 4.44 | 31 |
| 21.45 | 4.14 | 82 |
| 22.02 | 4.04 | 22 |
| 22.37 | 3.97 | 31 |
| 22.92 | 3.88 | 20 |
| 23.31 | 3.82 | 48 |
| 24.40 | 3.65 | 20 |
| 25.01 | 3.56 | 13 |
| 25.38 | 3.51 | 9 |
| 25.91 | 3.44 | 15 |
| 26.54 | 3.36 | 8 |
| 27.10 | 3.29 | 19 |
| 27.24 | 3.27 | 27 |
| 28.06 | 3.18 | 100 |
| 28.58 | 3.12 | 24 |

TABLE 7a
X-ray powder reflections (up to 30° 2Θ) and intensities (normalized) of
a fumarate salt of BIBR 1048 (form III)

| 2Θ [°] | d [Å] | I/Iₒ [%] |
|---|---|---|
| 3.14 | 28.12 | 100 |
| 4.54 | 19.46 | 19 |
| 5.50 | 16.07 | 12 |
| 6.86 | 12.89 | 7 |
| 7.34 | 12.04 | 9 |
| 8.26 | 10.70 | 9 |
| 8.84 | 10.01 | 16 |
| 9.21 | 9.60 | 21 |
| 9.85 | 8.98 | 32 |
| 10.45 | 8.47 | 19 |
| 10.71 | 8.26 | 16 |
| 11.57 | 7.65 | 11 |
| 11.92 | 7.42 | 18 |
| 12.37 | 7.15 | 14 |
| 12.66 | 6.99 | 0 |
| 13.34 | 6.64 | 15 |
| 13.67 | 6.48 | 18 |
| 14.50 | 6.11 | 9 |
| 15.48 | 5.72 | 43 |
| 16.62 | 5.33 | 10 |
| 16.94 | 5.24 | 15 |
| 17.29 | 5.13 | 19 |
| 17.62 | 5.03 | 10 |
| 18.04 | 4.92 | 9 |
| 19.57 | 4.54 | 22 |
| 20.14 | 4.41 | 7 |
| 20.94 | 4.24 | 10 |
| 21.54 | 4.13 | 13 |
| 22.34 | 3.98 | 11 |
| 22.66 | 3.92 | 11 |
| 23.27 | 3.82 | 12 |
| 24.02 | 3.71 | 10 |
| 24.85 | 3.58 | 20 |
| 25.10 | 3.55 | 15 |
| 25.46 | 3.50 | 17 |
| 25.75 | 3.46 | 27 |
| 26.86 | 3.32 | 14 |
| 27.09 | 3.29 | 20 |
| 28.30 | 3.15 | 8 |
| 28.78 | 3.10 | 9 |

TABLE 7b
X-ray powder reflections (up to 30° 2Θ) and intensities (normalized) of
a fumarate salt of BIBR 1048 (form IV)

| 2Θ [°] | d [Å] | I/Iₒ [%] |
|---|---|---|
| 3.69 | 23.92 | 100 |
| 5.70 | 15.50 | 13 |
| 7.37 | 11.99 | 23 |
| 8.58 | 10.30 | 25 |
| 9.43 | 9.38 | 25 |
| 9.94 | 8.90 | 3 |
| 11.90 | 7.44 | 11 |
| 12.32 | 7.18 | 18 |
| 12.98 | 6.82 | 4 |
| 14.06 | 6.30 | 20 |
| 15.02 | 5.90 | 11 |
| 15.81 | 5.60 | 10 |
| 17.17 | 5.16 | 15 |
| 17.40 | 5.09 | 16 |
| 17.80 | 4.98 | 45 |
| 18.51 | 4.79 | 15 |
| 18.95 | 4.68 | 14 |
| 19.14 | 4.64 | 8 |
| 19.76 | 4.49 | 26 |
| 20.49 | 4.33 | 31 |
| 20.90 | 4.25 | 10 |
| 21.46 | 4.14 | 5 |
| 22.01 | 4.04 | 23 |
| 22.30 | 3.99 | 11 |
| 22.72 | 3.91 | 14 |
| 23.26 | 3.82 | 4 |
| 23.90 | 3.72 | 34 |
| 24.30 | 3.66 | 8 |
| 24.68 | 3.61 | 19 |
| 25.09 | 3.55 | 38 |
| 25.53 | 3.49 | 26 |
| 25.90 | 3.44 | 6 |
| 26.26 | 3.39 | 10 |
| 26.50 | 3.36 | 11 |
| 26.72 | 3.34 | 13 |
| 27.04 | 3.30 | 12 |
| 27.54 | 3.24 | 4 |
| 28.18 | 3.17 | 8 |
| 28.42 | 3.14 | 9 |
| 28.78 | 3.10 | 8 |
| 29.14 | 3.06 | 5 |
| 29.79 | 3.00 | 7 |

TABLE 8
X-ray powder reflections (up to 30° 2Θ) and intensities (normalized) of
a glucuronate salt of BIBR 1048 (form I)

| 2Θ [°] | d [Å] | I/Iₒ [%] |
|---|---|---|
| 3.60 | 24.52 | 100 |
| 4.20 | 21.06 | 5 |
| 5.15 | 17.15 | 4 |
| 6.42 | 13.77 | 2 |
| 6.75 | 13.09 | 4 |
| 7.06 | 12.52 | 2 |
| 8.20 | 10.78 | 4 |
| 10.71 | 8.26 | 4 |
| 12.02 | 7.36 | 3 |
| 12.46 | 7.10 | 3 |
| 13.02 | 6.80 | 2 |
| 13.30 | 6.66 | 3 |
| 13.89 | 6.37 | 7 |
| 14.20 | 6.24 | 6 |
| 16.28 | 5.44 | 13 |
| 16.88 | 5.25 | 6 |
| 17.41 | 5.09 | 6 |
| 17.89 | 4.96 | 7 |
| 18.76 | 4.73 | 6 |
| 19.75 | 4.50 | 4 |
| 20.54 | 4.32 | 2 |
| 21.78 | 4.08 | 3 |
| 23.22 | 3.83 | 5 |
| 23.70 | 3.75 | 5 |
| 24.28 | 3.67 | 9 |
| 25.28 | 3.52 | 6 |
| 25.81 | 3.45 | 5 |
| 26.27 | 3.39 | 5 |
| 26.75 | 3.33 | 5 |
| 27.22 | 3.28 | 3 |
| 27.54 | 3.24 | 2 |

TABLE 9a
X-ray powder reflections (up to 30° 2Θ) and intensities (normalized) of
a glycolate salt of BIBR 1048 (form I)

| 2Θ [°] | d [Å] | I/Iₒ [%] |
|---|---|---|
| 5.09 | 17.36 | 100 |
| 7.48 | 11.81 | 6 |
| 8.46 | 10.45 | 4 |
| 10.32 | 8.57 | 23 |
| 11.72 | 7.54 | 4 |
| 12.87 | 6.88 | 9 |
| 14.16 | 6.25 | 13 |
| 14.80 | 5.98 | 15 |
| 16.37 | 5.41 | 7 |
| 16.86 | 5.26 | 7 |
| 17.82 | 4.97 | 3 |
| 18.73 | 4.73 | 9 |
| 19.48 | 4.55 | 12 |
| 20.31 | 4.37 | 33 |
| 20.84 | 4.26 | 18 |
| 21.44 | 4.14 | 23 |
| 21.84 | 4.07 | 16 |
| 22.27 | 3.99 | 7 |
| 22.69 | 3.92 | 6 |
| 23.65 | 3.76 | 49 |
| 24.38 | 3.65 | 7 |
| 25.53 | 3.49 | 9 |
| 26.00 | 3.42 | 9 |
| 26.44 | 3.37 | 6 |
| 27.21 | 3.27 | 3 |
| 27.89 | 3.20 | 10 |
| 28.83 | 3.09 | 15 |
| 29.56 | 3.02 | 4 |

TABLE 9b
X-ray powder reflections (up to 30° 2Θ) and intensities (normalized)
of a glycolate salt of BIBR 1048 (form II)

| 2Θ [°] | d [Å] | I/Iₒ [%] |
|---|---|---|
| 3.83 | 23.07 | 100 |
| 5.67 | 15.59 | 2 |
| 7.65 | 11.55 | 6 |
| 9.38 | 9.42 | 15 |
| 10.91 | 8.10 | 7 |
| 11.52 | 7.68 | 2 |
| 12.29 | 7.20 | 4 |
| 13.21 | 6.70 | 5 |
| 13.63 | 6.49 | 3 |
| 14.37 | 6.16 | 10 |
| 14.99 | 5.91 | 5 |
| 15.88 | 5.58 | 4 |
| 16.23 | 5.46 | 3 |
| 17.59 | 5.04 | 23 |
| 18.84 | 4.71 | 7 |
| 20.13 | 4.41 | 16 |
| 20.67 | 4.29 | 10 |
| 21.13 | 4.20 | 12 |
| 21.83 | 4.07 | 8 |
| 22.14 | 4.01 | 6 |
| 22.95 | 3.87 | 8 |
| 23.29 | 3.82 | 9 |
| 24.03 | 3.70 | 9 |
| 24.42 | 3.64 | 12 |
| 25.40 | 3.50 | 12 |
| 26.54 | 3.36 | 13 |
| 27.39 | 3.25 | 2 |
| 27.80 | 3.21 | 3 |
| 29.49 | 3.03 | 4 |

TABLE 9c
X-ray powder reflections (up to 30° 2Θ) and intensities (normalized)
of a glycolate salt of BIBR 1048 (form III)

| 2Θ [°] | d [Å] | I/Iₒ [%] |
|---|---|---|
| 3.66 | 24.14 | 100 |
| 7.27 | 12.16 | 1 |
| 10.61 | 8.33 | 16 |
| 11.55 | 7.66 | 4 |
| 12.00 | 7.37 | 3 |
| 12.69 | 6.97 | 2 |
| 14.81 | 5.98 | 6 |
| 15.25 | 5.80 | 5 |
| 16.88 | 5.25 | 4 |
| 17.35 | 5.11 | 3 |
| 18.32 | 4.84 | 10 |
| 19.37 | 4.58 | 8 |
| 20.03 | 4.43 | 16 |
| 20.85 | 4.26 | 11 |
| 21.50 | 4.13 | 4 |
| 22.95 | 3.87 | 6 |
| 23.25 | 3.82 | 9 |
| 23.52 | 3.78 | 7 |
| 24.42 | 3.64 | 12 |
| 24.71 | 3.60 | 11 |
| 25.78 | 3.45 | 3 |
| 26.77 | 3.33 | 6 |
| 27.49 | 3.24 | 3 |
| 27.75 | 3.21 | 2 |
| 28.67 | 3.11 | 1 |
| 29.53 | 3.02 | 2 |
| 30.41 | 2.94 | 2 |

TABLE 10
X-ray powder reflections (up to 30° 2Θ) and intensities (normalized) of
a isethionate salt of BIBR 1048 (form III)

| 2Θ [°] | d [Å] | I/Iₒ [%] |
|---|---|---|
| 3.54 | 24.97 | 100 |
| 4.31 | 20.52 | 86 |
| 5.86 | 15.09 | 26 |
| 7.04 | 12.56 | 21 |
| 7.67 | 11.53 | 87 |
| 8.58 | 10.31 | 15 |
| 9.38 | 9.43 | 4 |
| 10.50 | 8.42 | 26 |
| 11.66 | 7.59 | 41 |
| 13.14 | 6.74 | 23 |
| 13.44 | 6.59 | 27 |
| 14.02 | 6.32 | 6 |
| 14.42 | 6.14 | 4 |
| 14.82 | 5.98 | 8 |
| 15.30 | 5.79 | 35 |
| 16.77 | 5.29 | 78 |
| 17.52 | 5.06 | 44 |
| 18.19 | 4.88 | 20 |
| 18.86 | 4.71 | 23 |
| 19.58 | 4.53 | 6 |
| 19.81 | 4.48 | 19 |
| 20.08 | 4.42 | 5 |
| 20.91 | 4.25 | 46 |
| 21.70 | 4.10 | 14 |
| 22.02 | 4.04 | 9 |
| 22.46 | 3.96 | 10 |
| 22.97 | 3.87 | 27 |
| 23.90 | 3.72 | 27 |
| 24.83 | 3.59 | 20 |
| 25.42 | 3.50 | 11 |
| 25.66 | 3.47 | 11 |
| 26.50 | 3.36 | 10 |
| 26.82 | 3.32 | 12 |
| 27.14 | 3.29 | 11 |
| 27.65 | 3.23 | 22 |
| 28.10 | 3.18 | 5 |
| 28.50 | 3.13 | 6 |
| 28.82 | 3.10 | 4 |
| 29.38 | 3.04 | 4 |

TABLE 11
X-ray powder reflections (up to 30° 2Θ) and intensities (normalized) of
a L-malate salt of BIBR 1048 (form I)

| 2Θ [°] | d [Å] | I/Iₒ [%] |
|---|---|---|
| 3.03 | 29.18 | 100 |
| 5.12 | 17.24 | 29 |
| 5.54 | 15.95 | 18 |
| 5.90 | 14.98 | 9 |
| 7.78 | 11.36 | 3 |
| 9.01 | 9.81 | 12 |
| 9.72 | 9.10 | 25 |
| 12.04 | 7.35 | 20 |
| 12.85 | 6.89 | 27 |
| 13.10 | 6.76 | 9 |
| 14.22 | 6.23 | 6 |
| 15.26 | 5.81 | 11 |
| 15.84 | 5.59 | 10 |
| 16.61 | 5.34 | 23 |
| 17.71 | 5.01 | 14 |
| 18.06 | 4.91 | 16 |
| 18.50 | 4.80 | 7 |
| 19.50 | 4.55 | 32 |
| 20.22 | 4.39 | 10 |
| 20.62 | 4.31 | 8 |
| 21.34 | 4.16 | 9 |
| 22.06 | 4.03 | 4 |
| 22.57 | 3.94 | 9 |
| 22.89 | 3.88 | 9 |
| 23.67 | 3.76 | 10 |
| 24.37 | 3.65 | 27 |
| 24.70 | 3.60 | 24 |
| 25.82 | 3.45 | 10 |
| 26.28 | 3.39 | 10 |

TABLE 12
X-ray powder reflections (up to 30° 2Θ) and intensities (normalized) of
a D-malate salt of BIBR 1048 (form I)

| 2Θ [°] | d [Å] | I/Iₒ [%] |
|---|---|---|
| 3.08 | 28.69 | 100 |
| 5.14 | 17.20 | 11 |
| 5.50 | 16.07 | 8 |
| 5.86 | 15.08 | 4 |
| 9.05 | 9.78 | 7 |
| 9.74 | 9.08 | 11 |
| 12.08 | 7.33 | 9 |
| 12.90 | 6.86 | 8 |
| 14.26 | 6.21 | 4 |
| 15.30 | 5.79 | 7 |
| 15.78 | 5.62 | 7 |
| 16.63 | 5.33 | 10 |
| 17.70 | 5.01 | 8 |
| 18.03 | 4.92 | 8 |
| 18.54 | 4.79 | 3 |
| 19.48 | 4.56 | 11 |
| 20.22 | 4.39 | 2 |
| 21.22 | 4.19 | 4 |
| 21.70 | 4.10 | 4 |
| 22.86 | 3.89 | 5 |
| 23.71 | 3.75 | 8 |
| 24.34 | 3.66 | 10 |
| 24.78 | 3.59 | 9 |
| 25.83 | 3.45 | 7 |

TABLE 13
X-ray powder reflections (up to 30° 2Θ) and intensities (normalized) of
a S-(+)-mandelate salt of BIBR 1048 (form I)

| 2Θ [°] | d [Å] | I/Iₒ [%] |
|---|---|---|
| 3.02 | 29.29 | 10 |
| 5.04 | 17.52 | 100 |
| 6.03 | 14.67 | 12 |
| 8.92 | 9.92 | 10 |
| 11.10 | 7.97 | 12 |
| 11.29 | 7.84 | 13 |
| 11.54 | 7.67 | 7 |
| 13.18 | 6.72 | 19 |
| 14.45 | 6.13 | 20 |
| 15.39 | 5.76 | 12 |
| 16.27 | 5.45 | 18 |
| 16.70 | 5.31 | 6 |
| 17.88 | 4.96 | 20 |
| 18.51 | 4.79 | 15 |
| 19.18 | 4.63 | 4 |
| 20.41 | 4.35 | 17 |
| 20.82 | 4.27 | 14 |
| 21.53 | 4.13 | 20 |
| 23.22 | 3.83 | 10 |
| 23.76 | 3.74 | 12 |
| 24.42 | 3.65 | 5 |
| 24.86 | 3.58 | 7 |
| 25.14 | 3.54 | 10 |
| 25.66 | 3.47 | 11 |
| 26.50 | 3.36 | 9 |
| 27.02 | 3.30 | 11 |

TABLE 14
X-ray powder reflections (up to 30° 2Θ) and intensities (normalized) of
a naphthalene-1,5-disulfonate salt of BIBR 1048 (form I)

| 2Θ [°] | d [Å] | I/Iₒ [%] |
|---|---|---|
| 3.69 | 23.98 | 37 |
| 3.92 | 22.54 | 100 |
| 4.89 | 18.08 | 6 |
| 7.30 | 12.11 | 6 |
| 7.81 | 11.32 | 9 |
| 8.29 | 10.67 | 9 |
| 8.83 | 10.01 | 14 |
| 9.06 | 9.76 | 8 |
| 10.00 | 8.84 | 7 |
| 10.40 | 8.51 | 8 |
| 11.08 | 7.99 | 16 |
| 11.65 | 7.60 | 15 |
| 12.90 | 6.86 | 4 |
| 13.38 | 6.62 | 7 |
| 13.68 | 6.47 | 8 |
| 14.53 | 6.10 | 10 |
| 14.98 | 5.91 | 7 |
| 15.50 | 5.72 | 21 |
| 16.22 | 5.46 | 5 |
| 16.54 | 5.36 | 17 |
| 16.84 | 5.26 | 10 |
| 17.14 | 5.17 | 4 |
| 17.67 | 5.02 | 10 |
| 18.07 | 4.91 | 7 |
| 19.25 | 4.61 | 10 |
| 19.57 | 4.54 | 9 |
| 20.07 | 4.42 | 9 |
| 20.59 | 4.31 | 7 |
| 21.57 | 4.12 | 14 |
| 22.12 | 4.02 | 11 |
| 22.30 | 3.99 | 11 |
| 22.86 | 3.89 | 5 |
| 23.41 | 3.80 | 16 |
| 23.74 | 3.75 | 7 |
| 24.14 | 3.69 | 10 |
| 24.77 | 3.59 | 23 |
| 25.78 | 3.46 | 5 |
| 26.17 | 3.41 | 14 |
| 26.66 | 3.34 | 17 |
| 27.91 | 3.20 | 8 |

TABLE 15
X-ray powder reflections (up to 30° 2Θ) and intensities (normalized) of
a naphthalene-2-sulfonate salt of BIBR 1048 (form I)

| 2Θ [°] | d [Å] | I/Iₒ [%] |
|---|---|---|
| 4.23 | 20.89 | 100 |
| 5.66 | 15.62 | 9 |
| 7.42 | 11.91 | 5 |
| 8.16 | 10.83 | 21 |
| 9.14 | 9.68 | 5 |
| 11.22 | 7.89 | 4 |
| 11.88 | 7.45 | 5 |
| 12.14 | 7.29 | 5 |
| 12.70 | 6.97 | 4 |
| 13.05 | 6.78 | 5 |
| 13.42 | 6.60 | 5 |
| 13.66 | 6.48 | 6 |
| 14.74 | 6.01 | 4 |
| 15.02 | 5.90 | 4 |
| 15.30 | 5.79 | 4 |
| 15.64 | 5.66 | 4 |
| 16.81 | 5.27 | 13 |
| 17.27 | 5.13 | 12 |
| 17.98 | 4.93 | 11 |
| 18.38 | 4.83 | 13 |
| 18.90 | 4.70 | 4 |
| 20.34 | 4.37 | 3 |
| 21.10 | 4.21 | 5 |
| 21.52 | 4.13 | 6 |
| 22.49 | 3.95 | 12 |
| 23.18 | 3.84 | 4 |
| 23.94 | 3.72 | 8 |
| 24.21 | 3.68 | 7 |
| 24.78 | 3.59 | 6 |
| 25.06 | 3.55 | 8 |
| 25.53 | 3.49 | 6 |
| 25.91 | 3.44 | 4 |
| 26.62 | 3.35 | 4 |
| 27.42 | 3.25 | 4 |
| 27.82 | 3.21 | 3 |
| 28.38 | 3.14 | 3 |

TABLE 16a
X-ray powder reflections (up to 30° 2Θ) and intensities
(normalized) of a oxalate salt of BIBR 1048 (form I)

| 2Θ [°] | d [Å] | I/Iₒ [%] |
|---|---|---|
| 4.25 | 20.78 | 100 |
| 5.48 | 16.13 | 20 |
| 6.22 | 14.21 | 11 |
| 6.59 | 13.42 | 23 |
| 8.12 | 10.89 | 10 |
| 8.66 | 10.21 | 7 |
| 9.25 | 9.56 | 8 |
| 9.91 | 8.93 | 14 |
| 10.05 | 8.80 | 14 |
| 15.98 | 5.55 | 5 |
| 17.02 | 5.21 | 17 |
| 17.42 | 5.09 | 8 |
| 17.67 | 5.02 | 11 |
| 18.53 | 4.79 | 23 |
| 19.46 | 4.56 | 10 |
| 19.86 | 4.47 | 15 |
| 20.39 | 4.35 | 25 |
| 21.36 | 4.16 | 11 |
| 21.75 | 4.09 | 17 |
| 22.29 | 3.99 | 11 |
| 23.14 | 3.84 | 8 |
| 23.74 | 3.75 | 12 |
| 24.18 | 3.68 | 9 |
| 24.58 | 3.62 | 7 |
| 25.24 | 3.53 | 13 |
| 25.63 | 3.47 | 13 |
| 25.94 | 3.43 | 9 |
| 26.78 | 3.33 | 5 |
| 27.35 | 3.26 | 10 |
| 28.58 | 3.12 | 7 |
| 28.82 | 3.10 | 7 |
| 29.02 | 3.08 | 6 |
| 29.70 | 3.01 | 5 |

TABLE 16b
X-ray powder reflections (up to 30° 2Θ) and intensities
(normalized) of a oxalate salt of BIBR 1048 (form II)

| 2Θ [°] | d [Å] | I/Iₒ [%] |
|---|---|---|
| 3.33 | 26.51 | 100 |
| 6.65 | 13.29 | 25 |
| 9.78 | 9.04 | 3 |
| 10.74 | 8.24 | 15 |
| 11.02 | 8.03 | 17 |
| 12.27 | 7.22 | 22 |
| 12.46 | 7.10 | 20 |
| 13.42 | 6.60 | 14 |
| 13.78 | 6.43 | 7 |
| 14.69 | 6.03 | 14 |
| 15.82 | 5.60 | 5 |
| 16.22 | 5.46 | 4 |
| 16.82 | 5.27 | 6 |
| 17.78 | 4.99 | 4 |
| 19.38 | 4.58 | 38 |
| 19.68 | 4.51 | 34 |
| 20.49 | 4.33 | 31 |
| 21.72 | 4.09 | 35 |
| 22.15 | 4.01 | 21 |
| 22.84 | 3.89 | 34 |
| 23.42 | 3.80 | 6 |
| 23.80 | 3.74 | 15 |
| 24.29 | 3.66 | 32 |
| 25.19 | 3.54 | 22 |
| 26.09 | 3.41 | 19 |

TABLE 16c
X-ray powder reflections (up to 30° 2Θ) and intensities
(normalized) of a oxalate salt of BIBR 1048 (form V)

| 2Θ [°] | d [Å] | I/Iₒ [%] |
|---|---|---|
| 3.35 | 26.38 | 100 |
| 4.32 | 20.45 | 23 |
| 6.22 | 14.21 | 13 |
| 6.67 | 13.25 | 21 |
| 9.23 | 9.58 | 10 |
| 10.62 | 8.33 | 19 |
| 11.13 | 7.95 | 10 |
| 12.20 | 7.25 | 25 |
| 12.74 | 6.95 | 7 |
| 13.53 | 6.55 | 17 |
| 14.57 | 6.08 | 10 |
| 15.58 | 5.69 | 14 |
| 15.86 | 5.59 | 6 |
| 16.72 | 5.30 | 12 |
| 17.12 | 5.18 | 12 |
| 17.42 | 5.09 | 5 |
| 17.94 | 4.94 | 15 |
| 19.10 | 4.65 | 37 |
| 19.38 | 4.58 | 15 |
| 19.58 | 4.53 | 18 |
| 20.18 | 4.40 | 27 |
| 20.84 | 4.26 | 17 |
| 21.06 | 4.22 | 11 |
| 21.51 | 4.13 | 28 |
| 21.94 | 4.05 | 5 |
| 22.54 | 3.94 | 4 |
| 22.86 | 3.89 | 20 |
| 23.57 | 3.77 | 22 |
| 24.29 | 3.66 | 17 |
| 24.56 | 3.62 | 14 |
| 25.18 | 3.54 | 18 |
| 26.17 | 3.40 | 11 |
| 29.49 | 3.03 | 9 |

TABLE 17a
X-ray powder reflections (up to 30° 2Θ) and intensities
(normalized) of a phosphate salt of BIBR 1048 (form I)

| 2Θ [°] | d [Å] | I/Iₒ [%] |
|---|---|---|
| 3.90 | 22.63 | 100 |
| 5.96 | 14.82 | 9 |
| 9.06 | 9.76 | 3 |
| 9.89 | 8.94 | 12 |
| 10.73 | 8.24 | 8 |
| 11.18 | 7.91 | 10 |
| 12.58 | 7.04 | 6 |
| 12.89 | 6.87 | 14 |
| 13.67 | 6.48 | 9 |
| 14.18 | 6.25 | 4 |
| 14.85 | 5.96 | 16 |
| 15.14 | 5.85 | 10 |
| 15.74 | 5.63 | 8 |
| 16.37 | 5.41 | 10 |
| 16.74 | 5.30 | 5 |
| 17.10 | 5.18 | 3 |
| 17.42 | 5.09 | 5 |
| 18.24 | 4.86 | 28 |
| 18.86 | 4.70 | 3 |
| 19.57 | 4.54 | 8 |
| 20.11 | 4.42 | 9 |
| 20.79 | 4.27 | 16 |
| 21.63 | 4.11 | 17 |
| 22.14 | 4.02 | 12 |
| 22.57 | 3.94 | 12 |
| 23.50 | 3.79 | 6 |
| 23.98 | 3.71 | 12 |
| 24.29 | 3.66 | 13 |
| 25.55 | 3.49 | 8 |
| 25.95 | 3.43 | 8 |
| 26.42 | 3.37 | 4 |
| 26.86 | 3.32 | 4 |
| 27.27 | 3.27 | 8 |
| 27.82 | 3.21 | 9 |
| 28.75 | 3.11 | 8 |

TABLE 17b
X-ray powder reflections (up to 30° 2Θ) and intensities
(normalized) of a phosphate salt of BIBR 1048 (form II)

| 2Θ [°] | d [Å] | I/Iₒ [%] |
|---|---|---|
| 3.77 | 23.43 | 101 |
| 5.63 | 15.68 | 20 |
| 7.50 | 11.78 | 18 |
| 9.86 | 8.97 | 12 |
| 10.70 | 8.27 | 7 |
| 11.02 | 8.03 | 5 |
| 11.26 | 7.85 | 12 |
| 14.87 | 5.96 | 11 |
| 15.82 | 5.60 | 3 |
| 16.30 | 5.44 | 3 |
| 16.74 | 5.30 | 3 |
| 17.66 | 5.02 | 7 |
| 18.31 | 4.85 | 13 |
| 18.80 | 4.72 | 13 |
| 20.88 | 4.25 | 19 |
| 21.21 | 4.19 | 18 |
| 22.55 | 3.94 | 14 |
| 23.41 | 3.80 | 11 |
| 24.30 | 3.66 | 5 |
| 24.94 | 3.57 | 5 |
| 25.58 | 3.48 | 8 |
| 26.79 | 3.33 | 10 |

TABLE 18a
X-ray powder reflections (up to 30° 2Θ) and intensities
(normalized) of a propionate salt of BIBR 1048 (form I)

| 2Θ [°] | d [Å] | I/Iₒ [%] |
|---|---|---|
| 4.67 | 18.92 | 77 |
| 7.10 | 12.46 | 10 |
| 8.08 | 10.94 | 94 |
| 9.37 | 9.44 | 30 |
| 9.79 | 9.03 | 100 |
| 11.15 | 7.93 | 33 |
| 11.89 | 7.44 | 11 |
| 12.54 | 7.06 | 47 |
| 13.00 | 6.81 | 17 |
| 13.49 | 6.56 | 21 |
| 14.01 | 6.32 | 42 |
| 14.55 | 6.09 | 82 |
| 15.96 | 5.55 | 24 |
| 16.30 | 5.44 | 28 |
| 16.51 | 5.37 | 26 |
| 16.88 | 5.25 | 72 |
| 17.78 | 4.99 | 21 |
| 18.59 | 4.77 | 80 |
| 18.88 | 4.70 | 23 |
| 19.66 | 4.52 | 28 |
| 20.06 | 4.43 | 55 |
| 20.55 | 4.32 | 72 |
| 20.86 | 4.26 | 26 |
| 21.23 | 4.18 | 22 |
| 21.82 | 4.07 | 28 |
| 22.31 | 3.98 | 23 |
| 23.11 | 3.85 | 29 |
| 24.12 | 3.69 | 50 |
| 24.75 | 3.60 | 45 |
| 25.25 | 3.53 | 47 |
| 25.58 | 3.48 | 33 |
| 26.22 | 3.40 | 12 |
| 26.94 | 3.31 | 16 |
| 27.23 | 3.27 | 22 |
| 28.02 | 3.18 | 16 |
| 28.30 | 3.15 | 12 |
| 29.45 | 3.03 | 26 |

TABLE 18b
X-ray powder reflections (up to 30° 2Θ) and intensities
(normalized) of a propionate salt of BIBR 1048 (form II)

| 2Θ [°] | d [Å] | I/Iₒ [%] |
|---|---|---|
| 4.50 | 19.62 | 50 |
| 4.70 | 18.80 | 31 |
| 8.11 | 10.90 | 20 |
| 8.52 | 10.38 | 100 |
| 8.79 | 10.06 | 28 |
| 9.37 | 9.44 | 12 |
| 9.81 | 9.02 | 20 |
| 10.53 | 8.40 | 22 |
| 11.20 | 7.90 | 18 |
| 13.53 | 6.54 | 79 |
| 13.98 | 6.33 | 8 |
| 14.60 | 6.07 | 44 |
| 16.34 | 5.42 | 6 |
| 16.78 | 5.28 | 15 |
| 17.24 | 5.14 | 57 |
| 17.59 | 5.04 | 32 |
| 18.22 | 4.87 | 5 |
| 18.54 | 4.79 | 12 |
| 18.74 | 4.73 | 12 |
| 19.27 | 4.61 | 20 |
| 19.70 | 4.51 | 30 |
| 19.93 | 4.45 | 33 |
| 20.50 | 4.33 | 26 |
| 21.25 | 4.18 | 26 |
| 21.86 | 4.07 | 8 |
| 22.49 | 3.95 | 37 |
| 22.86 | 3.89 | 22 |
| 23.14 | 3.84 | 6 |
| 23.59 | 3.77 | 21 |
| 23.90 | 3.72 | 23 |
| 24.34 | 3.66 | 26 |
| 24.74 | 3.60 | 28 |
| 25.34 | 3.51 | 32 |
| 26.10 | 3.41 | 8 |
| 26.48 | 3.37 | 34 |
| 26.86 | 3.32 | 32 |
| 27.73 | 3.22 | 19 |
| 28.61 | 3.12 | 17 |
| 29.04 | 3.07 | 18 |

TABLE 19a
X-ray powder reflections (up to 30° 2Θ) and intensities
(normalized) of a saccharinate salt of BIBR 1048 (form I)

| 2Θ [°] | d [Å] | I/Iₒ [%] |
|---|---|---|
| 3.26 | 27.11 | 11 |
| 4.05 | 21.80 | 100 |
| 4.82 | 18.33 | 5 |
| 5.83 | 15.16 | 97 |
| 6.49 | 13.63 | 12 |
| 6.92 | 12.77 | 18 |
| 7.97 | 11.09 | 8 |
| 9.75 | 9.07 | 8 |
| 10.04 | 8.81 | 10 |
| 10.40 | 8.50 | 12 |
| 11.18 | 7.91 | 32 |
| 12.18 | 7.26 | 11 |
| 12.51 | 7.07 | 17 |
| 13.22 | 6.70 | 15 |
| 13.76 | 6.43 | 38 |
| 14.26 | 6.21 | 5 |
| 14.91 | 5.94 | 13 |
| 15.94 | 5.56 | 17 |
| 16.26 | 5.45 | 26 |
| 16.55 | 5.36 | 19 |
| 17.81 | 4.98 | 16 |
| 18.21 | 4.87 | 21 |
| 19.68 | 4.51 | 29 |
| 20.51 | 4.33 | 29 |
| 20.90 | 4.25 | 24 |
| 21.33 | 4.16 | 25 |
| 21.91 | 4.06 | 18 |
| 22.18 | 4.01 | 15 |
| 22.56 | 3.94 | 17 |
| 22.96 | 3.87 | 18 |
| 23.73 | 3.75 | 21 |
| 24.10 | 3.69 | 12 |
| 24.66 | 3.61 | 15 |
| 25.02 | 3.56 | 12 |
| 25.50 | 3.49 | 9 |
| 26.11 | 3.41 | 14 |
| 26.58 | 3.35 | 5 |
| 27.30 | 3.27 | 13 |

TABLE 19b
X-ray powder reflections (up to 30° 2Θ) and intensities
(normalized) of a saccharinate salt of BIBR 1048 (form II)

| 2Θ [°] | d [Å] | I/Iₒ [%] |
|---|---|---|
| 4.18 | 21.13 | 100 |
| 8.36 | 10.58 | 12 |
| 11.14 | 7.94 | 13 |
| 11.93 | 7.42 | 10 |
| 12.46 | 7.10 | 5 |
| 12.91 | 6.86 | 14 |
| 13.58 | 6.52 | 5 |
| 14.57 | 6.08 | 13 |
| 14.96 | 5.92 | 14 |
| 15.62 | 5.67 | 7 |
| 15.94 | 5.56 | 7 |
| 16.18 | 5.48 | 7 |
| 16.72 | 5.30 | 13 |
| 17.20 | 5.15 | 12 |
| 18.72 | 4.74 | 15 |
| 19.43 | 4.57 | 11 |
| 20.20 | 4.40 | 16 |
| 20.62 | 4.31 | 7 |
| 21.74 | 4.09 | 5 |
| 22.42 | 3.96 | 14 |
| 22.94 | 3.88 | 14 |
| 23.26 | 3.82 | 5 |
| 23.73 | 3.75 | 13 |
| 25.02 | 3.56 | 15 |
| 25.85 | 3.45 | 12 |
| 26.46 | 3.37 | 11 |

TABLE 19c
X-ray powder reflections (up to 30° 2Θ) and intensities (normalized)
of a saccharinate salt of BIBR 1048 (form III)

| 2Θ [°] | d [Å] | I/Iₒ [%] |
|---|---|---|
| 4.75 | 18.61 | 3 |
| 5.51 | 16.03 | 100 |
| 8.32 | 10.62 | 10 |
| 11.23 | 7.87 | 41 |
| 12.63 | 7.00 | 25 |
| 12.85 | 6.88 | 24 |
| 14.22 | 6.22 | 4 |
| 14.70 | 6.02 | 8 |
| 15.46 | 5.73 | 10 |
| 16.77 | 5.28 | 16 |
| 17.11 | 5.18 | 10 |
| 17.58 | 5.04 | 8 |
| 18.10 | 4.90 | 12 |
| 18.53 | 4.79 | 13 |
| 18.85 | 4.70 | 11 |
| 19.39 | 4.58 | 12 |
| 19.77 | 4.49 | 12 |
| 20.28 | 4.38 | 5 |
| 21.90 | 4.05 | 78 |
| 22.54 | 3.94 | 11 |
| 23.19 | 3.83 | 7 |
| 23.46 | 3.79 | 7 |
| 24.07 | 3.69 | 10 |
| 24.47 | 3.63 | 16 |
| 24.99 | 3.56 | 28 |
| 25.85 | 3.44 | 7 |
| 26.15 | 3.40 | 7 |
| 27.51 | 3.24 | 6 |
| 28.44 | 3.14 | 3 |

TABLE 19d
X-ray powder reflections (up to 30° 2Θ) and intensities (normalized)
of a saccharinate salt of BIBR 1048 (form IV)

| 2Θ [°] | d [Å] | I/Iₒ [%] |
|---|---|---|
| 5.55 | 15.92 | 100 |
| 6.91 | 12.79 | 12 |
| 8.74 | 10.10 | 2 |
| 11.19 | 7.90 | 35 |
| 12.15 | 7.28 | 15 |
| 13.03 | 6.79 | 7 |
| 13.54 | 6.54 | 24 |
| 14.42 | 6.14 | 6 |
| 15.36 | 5.76 | 8 |
| 16.47 | 5.38 | 27 |
| 17.63 | 5.03 | 8 |
| 18.77 | 4.72 | 42 |
| 19.72 | 4.50 | 20 |
| 21.12 | 4.20 | 3 |
| 21.88 | 4.06 | 56 |
| 22.33 | 3.98 | 34 |
| 23.32 | 3.81 | 23 |
| 23.92 | 3.72 | 18 |
| 24.88 | 3.58 | 34 |
| 26.07 | 3.41 | 13 |
| 26.66 | 3.34 | 7 |
| 27.35 | 3.26 | 5 |
| 27.77 | 3.21 | 6 |
| 29.08 | 3.07 | 4 |
| 30.02 | 2.97 | 2 |

TABLE 19e
X-ray powder reflections (up to 30° 2Θ) and intensities (normalized)
of a saccharinate salt of BIBR 1048 (form V)

| 2Θ [°] | d [Å] | I/Iₒ [%] |
|---|---|---|
| 4.99 | 17.70 | 73 |
| 6.22 | 14.20 | 100 |
| 7.51 | 11.76 | 60 |
| 9.54 | 9.26 | 8 |
| 9.89 | 8.93 | 8 |
| 10.19 | 8.67 | 6 |
| 11.09 | 7.97 | 11 |
| 11.55 | 7.66 | 9 |
| 11.84 | 7.47 | 9 |
| 12.24 | 7.23 | 5 |
| 12.64 | 7.00 | 6 |
| 13.40 | 6.60 | 5 |
| 14.06 | 6.30 | 19 |
| 14.90 | 5.94 | 29 |
| 16.00 | 5.54 | 21 |
| 17.43 | 5.08 | 37 |
| 18.88 | 4.70 | 25 |
| 19.85 | 4.47 | 40 |
| 20.89 | 4.25 | 13 |
| 22.55 | 3.94 | 28 |
| 23.52 | 3.78 | 11 |
| 25.14 | 3.54 | 37 |
| 25.88 | 3.44 | 25 |
| 26.68 | 3.34 | 23 |

TABLE 20a
X-ray powder reflections (up to 30° 2Θ) and intensities
(normalized) of a salicylate salt of BIBR 1048 (form II)

| 2Θ [°] | d [Å] | I/Iₒ [%] |
|---|---|---|
| 3.86 | 22.89 | 15 |
| 4.32 | 20.47 | 100 |
| 6.91 | 12.78 | 7 |
| 7.63 | 11.58 | 9 |
| 10.77 | 8.21 | 12 |
| 11.49 | 7.70 | 11 |
| 12.74 | 6.95 | 11 |
| 13.06 | 6.78 | 6 |
| 13.90 | 6.37 | 3 |
| 14.98 | 5.91 | 10 |
| 15.81 | 5.60 | 13 |
| 17.18 | 5.16 | 10 |
| 17.67 | 5.02 | 15 |
| 18.33 | 4.84 | 10 |
| 19.18 | 4.63 | 3 |
| 20.77 | 4.28 | 13 |
| 21.38 | 4.16 | 8 |
| 21.92 | 4.05 | 13 |
| 22.34 | 3.98 | 6 |
| 22.95 | 3.88 | 11 |
| 24.50 | 3.63 | 9 |
| 24.90 | 3.58 | 16 |
| 25.34 | 3.51 | 6 |
| 25.90 | 3.44 | 3 |
| 26.74 | 3.33 | 6 |
| 27.26 | 3.27 | 4 |
| 27.78 | 3.21 | 3 |
| 28.78 | 3.10 | 3 |
| 29.42 | 3.04 | 2 |

TABLE 20b
X-ray powder reflections (up to 30° 2Θ) and intensities
(normalized) of a salicylate salt of BIBR 1048 (form III)

| 2Θ [°] | d [Å] | I/Iₒ [%] |
|---|---|---|
| 4.41 | 20.04 | 100 |
| 7.44 | 11.88 | 4 |
| 11.47 | 7.71 | 6 |
| 13.08 | 6.77 | 9 |
| 14.01 | 6.32 | 7 |
| 14.85 | 5.96 | 8 |
| 15.76 | 5.62 | 7 |
| 17.56 | 5.05 | 11 |
| 18.53 | 4.78 | 4 |
| 20.14 | 4.41 | 5 |
| 20.60 | 4.31 | 7 |
| 21.38 | 4.15 | 6 |
| 21.95 | 4.05 | 7 |
| 23.92 | 3.72 | 6 |
| 24.43 | 3.64 | 11 |
| 24.74 | 3.60 | 11 |
| 25.68 | 3.47 | 18 |
| 26.79 | 3.33 | 9 |

TABLE 21a
X-ray powder reflections (up to 30° 2Θ) and intensities
(normalized) of a succinate salt of BIBR 1048 (form I)

| 2Θ [°] | d [Å] | I/Iₒ [%] |
|---|---|---|
| 4.52 | 19.56 | 17 |
| 7.54 | 11.71 | 1 |
| 8.30 | 10.65 | 10 |
| 9.23 | 9.58 | 70 |
| 10.66 | 8.29 | 35 |
| 11.93 | 7.41 | 34 |
| 12.58 | 7.03 | 27 |
| 13.25 | 6.68 | 37 |
| 13.80 | 6.41 | 55 |
| 14.77 | 5.99 | 23 |
| 15.50 | 5.71 | 93 |
| 16.56 | 5.35 | 6 |
| 17.37 | 5.10 | 56 |
| 18.07 | 4.91 | 14 |
| 19.28 | 4.60 | 51 |
| 19.94 | 4.45 | 95 |
| 21.01 | 4.23 | 61 |
| 21.35 | 4.16 | 69 |
| 22.60 | 3.93 | 52 |
| 24.05 | 3.70 | 29 |
| 24.94 | 3.57 | 65 |
| 26.25 | 3.39 | 12 |
| 27.16 | 3.28 | 100 |
| 28.03 | 3.18 | 18 |
| 28.92 | 3.08 | 8 |
| 29.78 | 3.00 | 4 |

TABLE 21b
X-ray powder reflections (up to 30° 2Θ) and intensities
(normalized) of a succinate salt of BIBR 1048 (form III)

| 2Θ [°] | d [Å] | I/Iₒ [%] |
|---|---|---|
| 3.10 | 28.45 | 100 |
| 5.30 | 16.66 | 17 |
| 8.51 | 10.39 | 14 |
| 9.18 | 9.63 | 20 |
| 9.84 | 8.99 | 40 |
| 10.71 | 8.26 | 10 |
| 12.08 | 7.32 | 13 |
| 13.10 | 6.75 | 12 |
| 13.74 | 6.44 | 11 |
| 14.45 | 6.13 | 8 |
| 15.54 | 5.70 | 16 |
| 16.78 | 5.28 | 21 |
| 17.33 | 5.11 | 15 |
| 17.79 | 4.98 | 20 |
| 19.13 | 4.64 | 11 |
| 19.69 | 4.50 | 28 |
| 20.30 | 4.37 | 14 |
| 21.50 | 4.13 | 14 |
| 22.62 | 3.93 | 8 |
| 23.21 | 3.83 | 9 |
| 24.97 | 3.56 | 17 |
| 25.90 | 3.44 | 24 |
| 27.16 | 3.28 | 19 |
| 28.24 | 3.16 | 7 |

TABLE 22
X-ray powder reflections (up to 30° 2Θ) and intensities (normalized) of
a D-tartrate salt of BIBR 1048 (form I)

| 2Θ [°] | d [Å] | I/Iₒ [%] |
|---|---|---|
| 3.20 | 27.62 | 100 |
| 3.78 | 23.37 | 25 |
| 4.21 | 20.97 | 8 |
| 4.82 | 18.35 | 20 |
| 5.14 | 17.19 | 15 |
| 9.27 | 9.54 | 13 |
| 12.08 | 7.33 | 12 |
| 12.68 | 6.98 | 10 |
| 13.07 | 6.77 | 9 |
| 14.91 | 5.94 | 4 |
| 15.86 | 5.59 | 5 |
| 17.24 | 5.14 | 13 |
| 18.55 | 4.78 | 17 |
| 19.42 | 4.57 | 12 |
| 21.66 | 4.10 | 7 |
| 24.81 | 3.59 | 12 |
| 25.76 | 3.46 | 15 |
| 26.70 | 3.34 | 8 |
| 28.42 | 3.14 | 6 |
| 29.41 | 3.04 | 5 |

TABLE 23
X-ray powder reflections (up to 30° 2Θ) and intensities (normalized) of
a D-tartrate salt of BIBR 1048 (form II)

| 2Θ [°] | d [Å] | I/Iₒ [%] |
|---|---|---|
| 3.10 | 28.49 | 100 |
| 4.01 | 22.06 | 52 |
| 4.87 | 18.14 | 25 |
| 5.50 | 16.07 | 10 |
| 6.07 | 14.56 | 8 |
| 7.98 | 11.08 | 10 |
| 9.10 | 9.72 | 12 |
| 9.29 | 9.52 | 18 |
| 9.91 | 8.92 | 19 |
| 10.54 | 8.39 | 5 |
| 11.22 | 7.89 | 10 |
| 11.38 | 7.77 | 9 |
| 12.07 | 7.33 | 16 |
| 12.38 | 7.15 | 13 |
| 13.12 | 6.75 | 16 |
| 14.22 | 6.23 | 6 |
| 15.07 | 5.88 | 19 |
| 16.67 | 5.32 | 22 |
| 17.06 | 5.20 | 5 |
| 17.46 | 5.08 | 5 |
| 18.20 | 4.87 | 34 |
| 18.73 | 4.74 | 26 |
| 19.10 | 4.65 | 10 |
| 20.62 | 4.31 | 17 |
| 21.16 | 4.20 | 19 |
| 21.70 | 4.10 | 6 |
| 22.11 | 4.02 | 15 |
| 22.95 | 3.87 | 18 |
| 23.42 | 3.80 | 15 |
| 23.82 | 3.74 | 8 |
| 24.26 | 3.67 | 12 |
| 24.87 | 3.58 | 22 |
| 25.14 | 3.54 | 17 |
| 25.49 | 3.49 | 23 |
| 25.86 | 3.44 | 12 |
| 27.09 | 3.29 | 8 |
| 28.30 | 3.15 | 5 |

TABLE 24a
X-ray powder reflections (up to 30° 2Θ) and intensities
(normalized) of a tosylate salt of BIBR 1048 (form I)

| 2Θ [°] | d [Å] | I/Iₒ [%] |
|---|---|---|
| 3.50 | 25.22 | 100 |
| 10.35 | 8.55 | 25 |
| 11.18 | 7.91 | 23 |
| 11.31 | 7.82 | 12 |
| 12.17 | 7.27 | 7 |
| 12.78 | 6.93 | 2 |
| 13.86 | 6.39 | 4 |
| 14.14 | 6.26 | 3 |
| 14.34 | 6.18 | 3 |
| 15.08 | 5.87 | 7 |
| 15.38 | 5.76 | 3 |
| 16.14 | 5.49 | 5 |
| 17.38 | 5.10 | 14 |
| 17.71 | 5.01 | 10 |
| 18.08 | 4.91 | 8 |
| 18.71 | 4.74 | 10 |
| 18.94 | 4.69 | 7 |
| 19.18 | 4.63 | 12 |
| 19.50 | 4.55 | 6 |
| 20.10 | 4.42 | 2 |
| 20.30 | 4.37 | 2 |
| 21.02 | 4.23 | 16 |
| 21.58 | 4.12 | 4 |
| 21.82 | 4.07 | 3 |
| 22.42 | 3.97 | 3 |
| 22.74 | 3.91 | 4 |
| 23.46 | 3.79 | 3 |
| 23.78 | 3.74 | 6 |
| 24.24 | 3.67 | 8 |
| 24.81 | 3.59 | 16 |
| 25.90 | 3.44 | 6 |
| 26.09 | 3.41 | 8 |
| 26.90 | 3.31 | 3 |
| 27.21 | 3.28 | 12 |
| 28.04 | 3.18 | 10 |
| 28.52 | 3.13 | 5 |
| 29.34 | 3.04 | 4 |
| 29.78 | 3.00 | 3 |

TABLE 24b
X-ray powder reflections (up to 30° 2Θ) and intensities
(normalized) of a tosylate salt of BIBR 1048 (form V)

| 2Θ [°] | d [Å] | I/Iₒ [%] |
|---|---|---|
| 4.15 | 21.30 | 100 |
| 8.29 | 10.67 | 10 |
| 8.54 | 10.35 | 7 |
| 10.00 | 8.85 | 13 |
| 10.54 | 8.39 | 8 |
| 10.94 | 8.09 | 3 |
| 11.96 | 7.40 | 7 |
| 12.90 | 6.86 | 2 |
| 13.26 | 6.68 | 2 |
| 13.90 | 6.37 | 2 |
| 14.26 | 6.21 | 2 |
| 15.03 | 5.89 | 9 |
| 15.70 | 5.64 | 4 |
| 16.17 | 5.48 | 9 |
| 16.97 | 5.22 | 11 |
| 17.42 | 5.09 | 11 |
| 17.80 | 4.98 | 12 |
| 18.30 | 4.85 | 5 |
| 18.81 | 4.72 | 9 |
| 19.06 | 4.66 | 6 |
| 19.85 | 4.47 | 10 |
| 20.02 | 4.43 | 8 |
| 20.54 | 4.32 | 15 |
| 20.86 | 4.26 | 8 |
| 21.19 | 4.19 | 12 |
| 21.96 | 4.05 | 10 |
| 22.50 | 3.95 | 6 |
| 22.98 | 3.87 | 10 |
| 23.18 | 3.84 | 8 |
| 24.22 | 3.67 | 6 |
| 25.06 | 3.55 | 9 |
| 25.82 | 3.45 | 5 |
| 27.12 | 3.29 | 9 |
| 27.54 | 3.24 | 7 |
| 27.98 | 3.19 | 4 |

TABLE 24c
X-ray powder reflections (up to 30° 2Θ) and intensities
(normalized) of a tosylate of BIBR 1048 (form VI)

| 2Θ [°] | d [Å] | I/Iₒ [%] |
|---|---|---|
| 4.25 | 20.78 | 34 |
| 8.51 | 10.38 | 14 |
| 8.93 | 9.9 | 14 |
| 9.19 | 9.61 | 10 |
| 11.23 | 7.87 | 9 |
| 11.44 | 7.73 | 21 |
| 11.83 | 7.48 | 49 |
| 12.23 | 7.23 | 39 |
| 13.37 | 6.62 | 14 |
| 14.44 | 6.13 | 37 |
| 15.22 | 5.82 | 59 |
| 16.5 | 5.37 | 7 |
| 17.69 | 5.01 | 34 |
| 17.91 | 4.95 | 73 |
| 18.14 | 4.89 | 77 |
| 18.43 | 4.81 | 24 |
| 18.62 | 4.76 | 41 |
| 19.04 | 4.66 | 87 |
| 19.47 | 4.56 | 68 |
| 19.72 | 4.5 | 52 |
| 20.27 | 4.38 | 22 |
| 21.19 | 4.19 | 100 |
| 22.01 | 4.04 | 29 |
| 22.24 | 3.99 | 14 |
| 22.58 | 3.93 | 82 |
| 22.95 | 3.87 | 43 |
| 23.26 | 3.82 | 37 |
| 23.75 | 3.74 | 36 |
| 24.58 | 3.62 | 11 |
| 25.51 | 3.49 | 25 |
| 25.98 | 3.43 | 12 |
| 26.32 | 3.38 | 5 |
| 26.58 | 3.35 | 10 |
| 26.98 | 3.3 | 14 |
| 27.32 | 3.26 | 15 |
| 27.77 | 3.21 | 9 |
| 28.68 | 3.11 | 41 |
| 28.92 | 3.08 | 26 |
| 29.3 | 3.05 | 15 |
| 29.82 | 2.99 | 10 |

TABLE 24d
X-ray powder reflections (up to 30° 2Θ) and intensities
(normalized) of a tosylate salt of BIBR 1048 (form VII)

| 2Θ [°] | d [Å] | I/Iₒ [%] |
|---|---|---|
| 3.47 | 25.44 | 100 |
| 10.37 | 8.52 | 13 |
| 11.16 | 7.92 | 8 |
| 11.76 | 7.52 | 4 |
| 13.36 | 6.62 | 3 |
| 14.04 | 6.30 | 3 |
| 15.31 | 5.78 | 1 |
| 16.04 | 5.52 | 2 |
| 16.47 | 5.38 | 2 |
| 17.60 | 5.04 | 12 |
| 18.84 | 4.71 | 10 |
| 19.41 | 4.57 | 4 |
| 20.69 | 4.29 | 8 |
| 22.25 | 3.99 | 4 |
| 24.29 | 3.66 | 14 |
| 26.52 | 3.36 | 9 |
| 27.16 | 3.28 | 4 |
| 28.24 | 3.16 | 3 |
| 29.29 | 3.05 | 2 |
| 29.60 | 3.02 | 2 |

### EXAMPLE A

Dry Ampoule Containing 75 mg Active Substance Per 10 ml

Composition:

| | | |
|---|---|---|
| active substance | 75.0 | mg |
| mannitol | 50.0 | mg |
| water for injections | ad 10.0 | ml |

Preparation:

Active substance and mannitol are dissolved in water. After packaging the solution is freeze-dried. To produce the solution ready for use for injections, the product is dissolved in water.

### EXAMPLE B

Dry ampoule containing 35 mg of active substance per 2 ml

Composition:

| | | |
|---|---|---|
| Active substance | 35.0 | mg |
| Mannitol | 100.0 | mg |
| water for injections | ad 2.0 | ml |

Preparation:

Active substance and mannitol are dissolved in water. After packaging, the solution is freeze-dried.

To produce the solution ready for use for injections, the product is dissolved in water.

### EXAMPLE C

Tablet containing 50 mg of active substance

Composition:

| | | |
|---|---|---|
| (1) Active substance | 50.0 | mg |
| (2) Lactose | 98.0 | mg |
| (3) Maize starch | 50.0 | mg |
| (4) Polyvinylpyrrolidone | 15.0 | mg |
| (5) Magnesium stearate | 2.0 | mg |
| | 215.0 | mg |

Preparation:

(1), (2) and (3) are mixed together and granulated with an aqueous solution of (4). (5) is added to the dried granulated material. From this mixture tablets are pressed, biplanar, faceted on both sides and with a dividing notch on one side.

Diameter of the tablets: 9 mm.

### EXAMPLE D

Tablet containing 350 mg of active substance

Composition:

| | | |
|---|---|---|
| (1) Active substance | 350.0 | mg |
| (2) Lactose | 136.0 | mg |
| (3) Maize starch | 80.0 | mg |
| (4) Polyvinylpyrrolidone | 30.0 | mg |
| (5) Magnesium stearate | 4.0 | mg |
| | 600.0 | mg |

Preparation:

(1), (2) and (3) are mixed together and granulated with an aqueous solution of (4). (5) is added to the dried granulated material. From this mixture tablets are pressed, biplanar, faceted on both sides and with a dividing notch on one side.

Diameter of the tablets: 12 mm.

### EXAMPLE E

Capsules containing 50 mg of active substance

Composition:

| | | |
|---|---|---|
| (1) Active substance | 50.0 | mg |
| (2) Dried maize starch | 58.0 | mg |
| (3) Powdered lactose | 50.0 | mg |
| (4) Magnesium stearate | 2.0 | mg |
| | 160.0 | mg |

Preparation:

(1) is triturated with (3). This trituration is added to the mixture of (2) and (4) with vigorous mixing.

This powder mixture is packed into size 3 hard gelatine capsules in a capsule filling machine.

### EXAMPLE F

Capsules containing 350 mg of active substance

Composition:

| | | |
|---|---|---|
| (1) Active substance | 350.0 | mg |
| (2) Dried maize starch | 46.0 | mg |
| (3) Powdered lactose | 30.0 | mg |
| (4) Magnesium stearate | 4.0 | mg |
| | 430.0 | mg |

Preparation:

(1) is triturated with (3). This trituration is added to the mixture of (2) and (4) with vigorous mixing.

This powder mixture is packed into size 0 hard gelatine capsules in a capsule filling machine.

### EXAMPLE G

Suppositories containing 100 mg of active substance

1 suppository contains:

| | | |
|---|---|---|
| Active substance | 100.0 | mg |
| Polyethyleneglycol (M.W. 1500) | 600.0 | mg |
| Polyethyleneglycol (M.W. 6000) | 460.0 | mg |
| Polyethylenesorbitan monostearate | 840.0 | mg |
| | 2,000.0 | mg |

### EXAMPLE H

| | Percentage composition | | | | | |
|---|---|---|---|---|---|---|
| | | | Active | | per | per |
| | Core | Separating | substance | | capsule | capsule |
| | material | layer | layer | Total | [mg] | [mg] |
| Tartaric acid | 61.3 | — | — | 61.3 | 176.7 | 353.4 |
| Gum arabic | 3.1 | 2.8 | | 5.9 | 17.0 | 34.0 |
| Talc | — | 5.6 | 3.2 | 8.8 | 25.4 | 50.7 |
| Hydroxyhydroxypropyl- | — | — | 4.0 | 4.0 | 11.5 | 23.1 |
| cellulose | | | | | | |
| Active substance (based | — | — | 20.0 | 20.0 | 50.0 | 100.0 |
| on the base) | | | | | | |
| Total | | | | 100.0 | 288.3 | 576.5 |

### EXAMPLE I

| | Percentage composition | | | | | |
|---|---|---|---|---|---|---|
| | | | Active | | per | per |
| | Core | Separating | substance | | capsule | capsule |
| | material | layer | layer | Total | [mg] | [mg] |
| Tartaric acid | 38.5 | — | — | 38.5 | 55.5 | 166.5 |
| Gum arabic | 1.9 | 1.7 | | 3.6 | 5.2 | 15.6 |
| Talc | — | 3.5 | 6.4 | 9.9 | 14.3 | 42.8 |
| Hydroxyhydroxypropyl- | — | — | 8.0 | 8.0 | 11.5 | 34.6 |
| cellulose | | | | | | |
| Active substance (based | — | — | 40.0 | 40.0 | 50.0 | 150.0 |
| on the base) | | | | | | |
| Total | | | | 100.0 | 144.2 | 432.5 |

The preparation and the structure of the pellets according to Examples H and I is described in detail in WO 03/074056.

1. A salt of ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate, wherein the salt is:
a) 2,5-dihydroxybenzoate,
b) besylate,
c) forms II, V and VI of the hydrochloride,
d) cyclamate,
e) edisylate,
f) esylate,
g) fumarate,
h) D-glucuronate,
i) glycolate,
j) isethionate,
k) L-malate,
l) D-malate,
m) mandelate,
n) naphthalene-1,5-disulfonate,
o) naphthalene-2-sulfonate,
p) oxalate,
q) phosphate,
r) propionate,
s) saccharinate,
t) forms II and III of the salicylate,
u) succinate,
v) D-tartrate,
w) tosylate, or
a polymorph or hydrate thereof.

2. The salt according to claim 1, wherein the salt is in crystalline form and is:
a) form II of the 2,5-dihydroxybenzoate,
b) forms I and II of the besylate,
c) forms II, V and VI of the hydrochloride,
d) form I of the cyclamate,
e) forms I and IV of the edisylate,
f) form I of the esylate,
h) form I of the D-glucuronate,
i) forms II and III of the glycolate,
j) form III of the isethionate,
k) form I of the L-malate,
l) form I of the D-malate,
m) form I of the mandelate,
n) form I of the naphthalene-1,5-disulfonate,
o) form I of the naphthalene-2-sulfonate,
p) forms I and V of the oxalate,
q) forms I and II of the phosphate,
s) forms I and II of the saccharinate,
t) form II of the salicylate,
u) form I of the succinate,
v) form I of the D-tartrate, and
w) forms I, V, VI and VII of the tosylate, or
a hydrate thereof.

3. The salt according to claim 2, wherein the salt is in crystalline form and is:
a) form II of the 2,5-dihydroxybenzoate,
b) forms I and II of the besylate,
f) form I of the esylate,
h) form I of the D-glucuronate,
k) form I of the L-malate,
l) form I of the D-malate,
s) forms I and II of the saccharinate,
t) form II of the salicylate,
u) form I of the succinate,
w) forms IV and VI of the tosylate, or
a hydrate thereof.

4-5. (canceled)

6. Forms II, V and VI of the hydrochloride salt of ethyl 3-[(2-[4-(hexyloxycarbonylamino-imino-methyl)-phenylamino]-methyl}-1-methyl-1H-benzimidazole-5-carbonyl)-pyridin-2-yl-amino]-propionate according to claim 2, wherein Forms II, V and VI are characterized by an X-ray powder diffraction pattern shown in FIGS. 3a, 3b and 3c, respectively.

7-21. (canceled)

22. Forms II and III of a salicylate salt of ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate and hydrates thereof, wherein Forms II and III are characterized by a melting point of 152° C. and 124° C., respectively.

23. (canceled)

24. Forms I and II of a D-tartrate salt of ethyl 3-[(2-[4-(hexyloxycarbonylamino-imino-methyl)-phenylamino]-methyl}-1-methyl-1H-benzimidazole-5-carbonyl)-pyridin-2-yl-amino]-propionate and hydrates thereof, wherein Forms I and II are charactered by the an X-ray powder diffraction pattern shown in FIGS. 22 and 23, respectively.

25. (canceled)

26. A method for prolonging activity of thrombin comprising administering to a patient in need thereof a pharmaceutically-acceptable amount of a salt according to claim 1.

27. A method for preventing venous thromboses and stroke comprising administering to a patient in need thereof a pharmaceutically-acceptable amount of a salt according to claim 1.

28. A pharmaceutical composition containing a salt according to claim 1, optionally together with one or more inert carriers and/or diluents.
